SEQUENCE LISTING (44 sequences)

SEQ ID NO: 1 - protein, 75 aa, unidentified
  source 1..75: note = coronavirus
MYSFVSEETGTLIVNSVLLFLAFVVFLLVTLAILTALRLCAYCCIVNVSLVKPTVYVYSRVKNLNSSEGVPDLLV

SEQ ID NO: 2 - protein, 16 aa, unidentified
  source 1..16: note = coronavirus
MYSFVSEETGTLIVNS

SEQ ID NO: 3 - protein, 16 aa, unidentified
  source 1..16: note = coronavirus
RVKNLNSSEGVPDLLV

SEQ ID NO: 4 - protein, 16 aa, unidentified
  source 1..16: note = coronavirus
NIVNVSLVKPTVYVYS

SEQ ID NO: 5 - protein, 15 aa, unidentified
  source 1..15: note = coronavirus
FLLVTLAILTALRLC

SEQ ID NO: 6 - protein, 32 aa, synthetic construct
  REGION 1..32: note = synthesized peptide
MYSFVSEETGTLIVNSRVKNLNSSEGVPDLLV

SEQ ID NO: 7 - protein, 12 aa, unidentified
  source 1..12: note = coronavirus
IKAYNPDEALLV

SEQ ID NO: 8 - protein, 12 aa, unidentified
  source 1..12: note = coronavirus
IKAYNPDGALLV

SEQ ID NO: 9 - protein, 12 aa, unidentified
  source 1..12: note = coronavirus
IKAYNPDGDLLV

SEQ ID NO: 10 - protein, 12 aa, unidentified
  source 1..12: note = coronavirus
IKAYNPDEAFLV

SEQ ID NO: 11 - protein, 12 aa, unidentified
  source 1..12: note = coronavirus
HIDPFPKRVIDF

SEQ ID NO: 12 - protein, 12 aa, unidentified
  source 1..12: note = coronavirus
RIDPLPSTVIDV

SEQ ID NO: 13 - protein, 12 aa, unidentified
  source 1..12: note = coronavirus
QIAPVPAEVLNV

SEQ ID NO: 14 - protein, 12 aa, unidentified
  source 1..12: note = coronavirus
LNSSEGVPDLLV

SEQ ID NO: 15 - protein, 12 aa, unidentified
  source 1..12: note = coronavirus
DSKPPLPPDEWV

SEQ ID NO: 16 - protein, 12 aa, unidentified
  source 1..12: note = coronavirus
DVKPPVLDVDDV

SEQ ID NO: 17 - protein, 12 aa, unidentified
  source 1..12: note = coronavirus
DEKPPVLDVDDV

SEQ ID NO: 18 - protein, 12 aa, unidentified
  source 1..12: note = coronavirus
EMRLPLLEVDDI

SEQ ID NO: 19 - protein, 12 aa, unidentified
  source 1..12: note = coronavirus
EHVIPSTLDDLI

SEQ ID NO: 20 - protein, 12 aa, unidentified
  source 1..12: note = coronavirus
NFQDVQRDKLYS

SEQ ID NO: 21 - protein, 12 aa, unidentified
  source 1..12: note = coronavirus
NEFPKNGWKNGC

SEQ ID NO: 22 - protein, 17 aa, synthetic construct
  REGION 1..17: note = modified peptide
CMYSFVSEETGTLIVNS

SEQ ID NO: 23 - protein, 17 aa, synthetic construct
  REGION 1..17: note = modified peptide
CRVKNLNSSEGVPDLLV

SEQ ID NO: 24 - protein, 17 aa, synthetic construct
  REGION 1..17: note = modified peptide
CNIVNVSLVKPTVYVYS

SEQ ID NO: 25 - protein, 17 aa, synthetic construct
  REGION 1..17: note = modified peptide
KKFLLVTLAILTALRLC

SEQ ID NO: 26 - protein, 33 aa, synthetic construct
  REGION 1..33: note = modified peptide
CMYSFVSEETGTLIVNSRVKNLNSSEGVPDLLV

SEQ ID NO: 27 - protein, 32 aa, synthetic construct
  REGION 1..32: note = modified peptide
CYSFVSEETGTLIVNSRVKNLNSSEGVPDLLV

SEQ ID NO: 28 - protein, 13 aa, synthetic construct
  REGION 1..13: note = modified peptide
CIKAYNPDEALLV

SEQ ID NO: 29 - protein, 13 aa, synthetic construct
  REGION 1..13: note = modified peptide
CIKAYNPDGALLV

SEQ ID NO: 30 - protein, 13 aa, synthetic construct
  REGION 1..13: note = modified peptide
CIKAYNPDGDLLV

SEQ ID NO: 31 - protein, 13 aa, synthetic construct
  REGION 1..13: note = modified peptide
CIKAYNPDEAFLV

SEQ ID NO: 32 - protein, 13 aa, synthetic construct
  REGION 1..13: note = modified peptide
CHIDPFPKRVIDF

SEQ ID NO: 33 - protein, 13 aa, synthetic construct
  REGION 1..13: note = modified peptide
CRIDPLPSTVIDV

SEQ ID NO: 34 - protein, 13 aa, synthetic construct
  REGION 1..13: note = modified peptide
CQIAPVPAEVLNV

SEQ ID NO: 35 - protein, 13 aa, synthetic construct
  REGION 1..13: note = modified peptide
CLNSSEGVPDLLV

SEQ ID NO: 36 - protein, 13 aa, synthetic construct
  REGION 1..13: note = modified peptide
CDSKPPLPPDEWV

SEQ ID NO: 37 - protein, 13 aa, synthetic construct
  REGION 1..13: note = modified peptide
CDVKPPVLDVDDV

SEQ ID NO: 38 - protein, 13 aa, synthetic construct
  REGION 1..13: note = modified peptide
CEVKPPVLDVDDV

SEQ ID NO: 39 - protein, 13 aa, synthetic construct
  REGION 1..13: note = modified peptide
CEMRLPLLEVDDI

SEQ ID NO: 40 - protein, 13 aa, synthetic construct
  REGION 1..13: note = modified peptide
CEHVIPSTLDDLI

SEQ ID NO: 41 - protein, 13 aa, synthetic construct
  REGION 1..13: note = modified peptide
CNFQDVQRDKLYS

SEQ ID NO: 42 - protein, 13 aa, synthetic construct
  REGION 1..13: note = modified peptide
CNEFPKNGWKNGC

SEQ ID NO: 43 - protein, 32 aa, synthetic construct; identical to SEQ ID NO: 6
  REGION 1..32: note = modified peptide
  SITE 1: note = MISC_FEATURE - modified by R1 group
  SITE 32: note = MISC_FEATURE - modified by R2 group

SEQ ID NO: 44 - protein, 32 aa, synthetic construct; identical to SEQ ID NO: 6
  REGION 1..32: note = modified peptide
  SITE 1: note = MISC_FEATURE - modified by acetyl group
  SITE 32: note = MISC_FEATURE - modified by NH2 group

(19) United States
(12) Patent Application Publication
(10) Pub. No.: US 2024/0132576 A1
(43) Pub. Date: Apr. 25, 2024

(54) CORONAVIRUS: EARLY DETECTION AND TREATMENT

(71) Applicants: Ruey J YU, Chalfont, PA (US); Sachin K. VYAS, Edison, NJ (US)

(72) Inventors: Ruey J. YU, Chalfont, PA (US); Sachin K. VYAS, Edison, NJ (US)

(21) Appl. No.: 18/174,939

(22) Filed: Feb. 27, 2023

Related U.S. Application Data
(63) Continuation of application No. 17/653,207, filed on Mar. 2, 2022, now abandoned.
Application No. 17/653,207 is a continuation of application No. PCT/US21/23390, filed on Mar. 22, 2021.
(60) Provisional application No. 62/992,264, filed on Mar. 20, 2020.

(51) Int. Cl. C07K 16/10; A61K 39/215; G01N 33/569

(52) U.S. Cl. CPC C07K 16/10; A61K 39/215; G01N 33/56983; G01N 2333/165; G01N 2469/10; G01N 2800/26

(57) ABSTRACT

The application relates to certain new epitope peptides associated with coronavirus envolop protein and the use thereof. In particular, the application describes the antibodies and vaccines develped against these peptides, and the use thereof for the detection and treatment of coronaviral infection in human subjects.

### CROSS-REFERENCE TO RELATED APPLICATIONS

This application is a continuation of U.S. patent application Ser. No. 17/653,207 filed Mar. 2, 2022, which is a continuation of PCT/US2021/023990 filed Mar. 22, 2021, which claims priority under 35 U.S.C. § 119(b) to U.S. Provisional Patent Application No. 62/992,264, filed on Mar. 20, 2020, the disclosures of which is incorporated herein by reference in its entirety.

### FIELD OF THE INVENTION

The invention relates to a discovery of certain new epitope peptides and the antibodies and vaccines produced against the envelope protein associated with coronavirus, for the detection and treatment of coronaviral infection in human subjects.

### REFERENCE TO SEQUENCE LISTING SUBMITTED ELECTRONICALLY

This application contains a sequence listing, which is submitted electronically via EFS-Web as an XML formatted sequence listing with a file name “065813_37US3 Sequence Listing” and a creation date of Jul. 25, 2023 and having a size of 53 KB. The sequence listing submitted via EFS-Web is part of the specification and is herein incorporated by reference in its entirety.

### BACKGROUND OF THE INVENTION

A research article entitled “SHORT PEPTIDES VACCINES FOR EARLY DECTECTION AND TREATMENT OF CORONAIRUS” by Dr Ruey J. Yu was published on Apr. 27, 2020 more than one month after the filing date, Mar. 20, 2020, of the above-mentioned Provisional Patent Application.

Some other references regarding coronavirus are listed as follows:
1. Schoeman D. and Fielding B. C. (2019) Coronavirus Envelope Protein: Current Knowledge. Virology Journal (16): 69-208.
2. Levinson W. (2016) Viral Vaccines. Review of Medical Microbiology and Immunology. 280-284.
3. Doan T., Melvold R., Viselli S., and Waltenbaugh C. (2013) Immune Pharmacotherapy. Immunology. 283-297.
4. O'Hagan D. T. (2000) Transcutaneous Immunization Vaccine Adjuvants: Preparation Methods and Research Protocols. 315-326.

Researchers in the United States and Taiwan have demonstrated the potential of a novel protein-peptide vaccine to protect against infection with severe acute respiratory syndrome coronavirus 2 (SARS-CoV-2), the pathogen that causes coronavirus disease 2019 (COVID-19) based on bioRxiv preprint doi (World Wide Web at doi.org/10.1101/2020.11.30.399154); this version posted Nov. 30, 2020.

Coronaviruses are a type of virus and are also known as CoVs. Coronavirus primarily infects birds and animals, but recently have been shown to infect human. There are many different kinds of coronaviruses. Some of them can cause colds or other mild respiratory illnesses (nose, throat, lung), and some can cause more serious diseases, including severe acute respiratory syndrome (SARS), COVID-19, and Middle East respiratory syndrome (MERS).

Coronavirus is an RNA virus with a protein envelope that is required for its infection into the host cells (Schoeman and Fielding, Virology Journal, 2019, (16): 69-208). The CoV envelope protein is a short chain membrane protein of 76-109 amino acids with 8.4 to 12 kDa in size. There are three sections in the membrane protein, i.e., the N-Terminal, transmembrane, and C-Terminal of the protein. The N-terminal region contains hydrophilic 7-12 amino acids, followed by a large hydrophobic transmembrane domain of about 25 amino acids, and ending with a long hydrophilic carboxyl region of about 44-72 amino acids as shown below:

(SEQ ID NO: 1)
MYSFVSEETGTLIVNSVLLFLAFVVFLLVTLAILT
ALRLCAYCCIVNVSLVKPTVYVYSRVKNLNSSEGV
PDLLV.

Some companies developed coronavirus vaccines based on adenocarcinomas cancer. However, during the vaccination test, some human subjects developed severe neurological disorders, and further tests had to be discontinued. At present, massive national vaccination is going on with the vaccine developed from RNA of coronavirus. The only major side effect is known to be the hypersensitivity. However, sudden mysterious deaths including a doctor have been reported recently. Therefore, absolutely safe vaccines are needed if everyone in the U.S.A. is required to be vaccinated.

It is well known that vaccines produced from short peptides are the safest based on recent research on melanoma studies, and thus there is a need to develop effective and safe means for early detection and treatment of coronavirus infection based on short peptides.

### SUMMARY OF THE INVENTION

The present application satisfies this need by providing novel and unique ways for early detection and treatment of coronavirus infection without side effects.

In one general aspect, the present application relates to an isolated peptide consisting of an amino acid sequence selected from the group consisting of:

(SEQ ID NO: 2)
MYSFVSEETGTLIVNS,
(SEQ ID NO: 3)
RVKNLNSSEGVPDLLV,
(SEQ ID NO: 4)
NIVNVSLVKPTVYVYS,
(SEQ ID NO: 5)
FLLVTLAILTALRLC,
(SEQ ID NO: 6)
MYSFVSEETGTLIVNSRV
KNLNSSEGVPDLLV,
(SEQ ID NO: 7)
IKAYNPDEALLV,
(SEQ ID NO: 8)
IKAYNPDGALLV,
(SEQ ID NO: 9)
IKAYNPDGDLLV,
(SEQ ID NO: 10)
IKAYNPDEAFLV,
(SEQ ID NO: 11)
HIDPFPKRVIDF,
(SEQ ID NO: 12)
RIDPLPSTVIDV,
(SEQ ID NO: 13)
QIAPVPAEVLNV,
(SEQ ID NO: 14)
LNSSEGVPDLLV,
(SEQ ID NO: 15)
DSKPPLPPDEWV,
(SEQ ID NO: 16)
DVKPPVLDVDDV,
(SEQ ID NO: 17)
DEKPPVLDVDDV,
(SEQ ID NO: 18)
EMRLPLLEVDDI,
(SEQ ID NO: 19)
EHVIPSTLDDLI,
(SEQ ID NO: 20)
NFQDVQRDKLYS,
(SEQ ID NO: 21)
NEFPKNGWKNGC,
(SEQ ID NO: 22)
CMYSFVSEETGTLIVNS,
(SEQ ID NO: 23)
CRVKNLNSSEGVPDLLV,
(SEQ ID NO: 24)
CNIVNVSLVKPTVYVYS,
(SEQ ID NO: 25)
KKFLLVTLAILTALRLC,
(SEQ ID NO: 26)
CMYSFVSEETGTLIVNSR
VKNLNSSEGVPDLLV,
(SEQ ID NO: 27)
CYSFVSEETGTLIVNSR
VKNLNSSEGVPDLLV,
(SEQ ID NO: 28)
CIKAYNPDEALLV,
(SEQ ID NO: 29)
CIKAYNPDGALLV,
(SEQ ID NO: 30)
CIKAYNPDGDLLV,
(SEQ ID NO: 31)
CIKAYNPDEAFLV,
(SEQ ID NO: 32)
CHIDPFPKRVIDF,
(SEQ ID NO: 33)
CRIDPLPSTVIDV,
(SEQ ID NO: 34)
CQIAPVPAEVLNV,
(SEQ ID NO: 35)
CLNSSEGVPDLLV,
(SEQ ID NO: 36)
CDSKPPLPPDEWV,
(SEQ ID NO: 37)
CDVKPPVLDVDDV,
(SEQ ID NO: 38)
CEVKPPVLDVDDV,
(SEQ ID NO: 39)
CEMRLPLLEVDDI,
(SEQ ID NO: 40)
CEHVIPSTLDDLI,
(SEQ ID NO: 41)
CNFQDVQRDKLYS,
and
(SEQ ID NO: 42)
CNEFPKNGWKNGC;

or a derivative peptide thereof.

In some embodiments, the isolated peptide consists of an amino acid sequence selected from the group consisting of:

(SEQ ID NO: 2)
MYSFVSEETGTLIVNS,
(SEQ ID NO: 3)
RVKNLNSSEGVPDLLV,
(SEQ ID NO: 4)
NIVNVSLVKPTVYVYS,
(SEQ ID NO: 5)
FLLVTLAILTALRLC,
(SEQ ID NO: 6)
MYSFVSEETGTLIVNSRV
KNLNSSEGVPDLLV,
(SEQ ID NO: 22)
CMYSFVSEETGTLIVNS,
(SEQ ID NO: 23)
CRVKNLNSSEGVPDLLV,
(SEQ ID NO: 24)
CNIVNVSLVKPTVYVYS,
(SEQ ID NO: 25)
KKFLLVTLAILTALRLC,
(SEQ ID NO: 26)
CMYSFVSEETGTLIVNSRV
KNLNSSEGVPDLLV,
and
(SEQ ID NO: 27)
CYSFVSEETGTLIVNSRVK
NLNSSEGVPDLLV.

In some embodiments, the isolated peptide consists of an amino acid sequence selected from the group consisting of:

(SEQ ID NO: 2)
MYSFVSEETGTLIVNS,
(SEQ ID NO: 3)
RVKNLNSSEGVPDLLV,
(SEQ ID NO: 6)
MYSFVSEETGTLIVNSRVKNLNSSEGVPDLLV,
(SEQ ID NO: 22)
CMYSFVSEETGTLIVNS,
(SEQ ID NO: 23)
CRVKNLNSSEGVPDLLV,
(SEQ ID NO: 26)
CMYSFVSEETGTLIVNSRVKNLNSSEGVPDLLV,
and
(SEQ ID NO: 27)
CYSFVSEETGTLIVNSRVKNLNSSEGVPDLLV.

In some embodiments, the isolated peptide consists of an amino acid sequence selected from the group consisting of:

(SEQ ID NO: 7)
IKAYNPDEALLV,
(SEQ ID NO: 8)
IKAYNPDGALLV,
(SEQ ID NO: 9)
IKAYNPDGDLLV,
(SEQ ID NO: 10)
IKAYNPDEAFLV,
(SEQ ID NO: 11)
HIDPFPKRVIDF,
(SEQ ID NO: 12)
RIDPLPSTVIDV,
(SEQ ID NO: 13)
QIAPVPAEVLNV,
(SEQ ID NO: 14)
LNSSEGVPDLLV,
(SEQ ID NO: 15)
DSKPPLPPDEWV,
(SEQ ID NO: 16)
DVKPPVLDVDDV,
(SEQ ID NO: 17)
DEKPPVLDVDDV,
(SEQ ID NO: 18)
EMRLPLLEVDDI,
(SEQ ID NO: 19)
EHVIPSTLDDLI,
(SEQ ID NO: 20)
NFQDVQRDKLYS,
(SEQ ID NO: 21)
NEFPKNGWKNGC,
(SEQ ID NO: 28)
CIKAYNPDEALLV,
(SEQ ID NO: 29)
CIKAYNPDGALLV,
(SEQ ID NO: 30)
CIKAYNPDGDLLV,
(SEQ ID NO: 31)
CIKAYNPDEAFLV,
(SEQ ID NO: 32)
CHIDPFPKRVIDF,
(SEQ ID NO: 33)
CRIDPLPSTVIDV,
(SEQ ID NO: 34)
CQIAPVPAEVLNV,
(SEQ ID NO: 35)
CLNSSEGVPDLLV,
(SEQ ID NO: 36)
CDSKPPLPPDEWV,
(SEQ ID NO: 37)
CDVKPPVLDVDDV,
(SEQ ID NO: 38)
CEVKPPVLDVDDV,
(SEQ ID NO: 39)
CEMRLPLLEVDDI,
(SEQ ID NO: 40)
CEHVIPSTLDDLI,
(SEQ ID NO: 41)
CNFQDVQRDKLYS,
and
(SEQ ID NO: 42)
CNEFPKNGWKNGC.

In another general aspect, the present application relates to a pharmaceutical composition, such as a vaccine or an immunogenic composition, comprising a peptide and a carrier protein, wherein the peptide consists of an amino acid sequence selected from the group consisting of SEQ IDs NO:2˜42 or a derivative peptide thereof.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or maltose binding protein (MBP), preferably KLH.

In another general aspect, the present application relates to a method of developing antibodies against coronavirus by administering a pharmaceutical composition, such as a vaccine or an immunogenic composition, to an animal or human, wherein the immunogenic composition comprises a peptide and a carrier protein, and wherein the peptide consists of an amino acid sequence selected from the group consisting of SEQ IDs NO:2˜42 or a derivative peptide thereof.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or maltose binding protein (MBP), preferably KLH.

In some embodiments, the animal used for developing antibodies against coronavirus is selected from the group consisting of rabbit, dog, monkey, and chimpanzee, preferably rabbit.

In some embodiments, the method comprises administering apharmaceutical composition, such as the vaccine or the immunogenic composition, according to an embodiment of the application to a human.

In some embodiments, the antibodies are polyclonal antibodies.

In another general aspect, the present application relates to an antibody against coronavirus, wherein the antibodies are developed by administering a pharmaceutical composition, such as a vaccine or an immunogenic composition, to an animal or human, wherein the pharmaceutical composition comprises a peptide and a carrier protein, and wherein the peptide consists of an amino acid sequence selected from the group consisting of SEQ IDs NO:2˜42 or a derivative peptide thereof.

In some embodiments, the peptide consists of an amino acid sequence selected from the group consisting of SEQ IDs NO:2˜6 and SEQ IDs NO:22-27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or MBP, preferably KLH.

In some embodiments, the animal is selected from the group consisting of rabbit, dog, monkey, and chimpanzee, preferably rabbit.

In some embodiments, the method comprises administering the pharmaceutical composition to human.

In some embodiments, the antibody is a polyclonal antibody.

In another general aspect, the present application relates to a method of detecting coronavirus in a subject in need thereof, the method comprising:
a. obtaining a sample from the subject; and
b. detecting in the sample the presence of one or more antibodies targeting a peptide, or the presence of one or more antigens that bind specifically to the antibody, wherein the peptide consists of an amino acid sequence selected from the group consisting of SEQ IDs NO:2˜42 or a derivative peptide thereof.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the sample is saliva or serum.

In some embodiments, the antibodies is polyclonal antibodies.

In some embodiments, the antibodies are produced in animal, preferably selected from the group consisting of rabbit, dog, monkey, and chimpanzee, more preferably rabbit.

In some embodiments, the antibodies are produced in human.

In some embodiments, the antibodies are detected by an enzyme-linked immunosorbent assay (ELISA).

In some embodiments, the ELISA is direct ELISA, indirect ELISA, sandwich ELISA, or competitive ELISA.

In some embodiments, the subject has no symptom of coronavirus infection at the time of the detection of the coronavirus.

In some embodiments, the coronavirus is selected from the group consisting of SARS-CoV-2, SARS virus, MERS virus, and common cold virus.

In another general aspect, the present application relates to a method of treating a coronavirus infection in a subject in need thereof, the method comprising administering to the subject a pharmaceutical composition, such as a vaccine or an immunogenic composition, comprising a peptide and a carrier protein, and wherein the peptide consists of an amino acid sequence selected from the group consisting of SEQ IDs NO:2˜42 or a derivative peptide thereof.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, and MBP, preferably KLH.

In some embodiments, the subject has no symptom of coronavirus infection at the time of the treatment.

In some embodiments, the coronavirus is selected from the group consisting of SARS-CoV-2, SARS virus, MERS virus, and common cold virus.

In yet another general aspect, the present application relates to a method of treating a coronavirus infection in a subject in need thereof, the method comprising administering to the subject an antibody against coronavirus.

In some embodiments, the antibody is developed by administering a pharmaceutical composition, such as a vaccine or an immunogenic composition, to an animal or human, wherein the pharmaceutical composition comprises a peptide and a carrier protein, and wherein the peptide consists of an amino acid sequence selected from the group consisting of SEQ IDs NO:2˜42 or a derivative peptide thereof.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or MBP, preferably KLH.

In some embodiments, the subject has no symptom of coronavirus infection at the time of the treatment.

In some embodiments, the coronavirus is selected from the group consisting of SARS-CoV-2, SARS virus, MERS virus, and common cold virus.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1. shows the HPLC of the peptide of SEQ ID NO: 27.

FIG. 2. shows the mass spectrum of the peptide of SEQ ID NO: 27.

FIG. 3. shows dot blot testing data of purified antibodies against the peptide of SEQ ID NO: 27.

FIGS. 4A-D. show the HPLC of the peptide of SEQ ID NO: 28 (FIG. 4A), SEQ ID NO: 29 (FIG. 4B), SEQ ID NO: 30 (FIG. 4C), and SEQ ID NO: 31 (FIG. 4D).

FIGS. 5A-D. show the mass spectra of the peptide of SEQ ID NO: 28 (FIG. 5A), SEQ ID NO: 29 (FIG. 5B), SEQ ID NO: 30 (FIG. 5C), and SEQ ID NO: 31 (FIG. 5D).

FIGS. 6A-D. show dot blot testing data of purified antibodies against the peptide of SEQ ID NO: 28 (FIG. 6A), SEQ ID NO: 29 (FIG. 6B), SEQ ID NO: 30 (FIG. 6C), and SEQ ID NO: 31 (FIG. 6D).

### DETAILED DESCRIPTION OF THE INVENTION

Unless defined otherwise, all technical and scientific terms used herein have the same meaning as commonly understood by one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Common or certain knowledge, scientific and medical terminologies can be readily found via internet, textbooks of chemistry, biochemistry, medicinal chemistry, pharmacology, dermatology and general medicine. The following are some examples. Robert K. Murray et al. eds. “Harper's Illustrated Biochemistry” 26th edn. Vol. I-II, McGraw Hill, 2003. Laurence L. Brunton et al. eds. “Goodman & Gilman's The Pharmacological Basis of Therapeutics” 12th edn. McGraw Hill Medical, 2011. Anthony S. Fauci et al. eds. “Harrison's Principles of Internal Medicine” 17th edn, McGraw Hill Medical, New York, 2008. Abba J. Kastin, Ed “Handbook of Biologically Active Peptides” 2nd edn. Academic Press, 2013. John Howl and Sarah Jones Ed “Bioactive peptides”, CRC Press 2009.

An amino acid is an organic acid having one or more than one alkaline radicals such as amino, guanidino, imino, or hydrazine radical attached at any carbon atom other than carbon one. There are 20 common amino acids which are represented by chemical names, such as “glycine”, or abbreviated symbols such as three letters, “Gly” or one letter “G. In this disclosure, both one letter and three letters will be used. Except glycine, all other common amino acids have stereoisomers, i.e., enantiomer, D or L form. The amino acids in most natural peptides and proteins are all in L-form. Some D-form amino acids are produced by microorganism or present in antibiotics, and have inhibitory or antagonistic actions. For example, D-alanine, D-aspartic acid, and D-glutamic acid are present in bacterial cell walls, and D-glutamic acid, D-aspartic acid and D-phenylalanine are present in antibiotic bacitracin. An uncommon amino acid is an amino acid that is not a common amino acid. Examples of uncommon amino acids include, but are not limited to, β-alanine and taurine. The uncommon amino acids can exist as a D or L form.

The one letter and three letter symbols used for the 20 common amino acids are as follows: alanine (A, Ala), arginine (R, Arg), aspartic acid (D, Asp), asparagine (N, Asn), cysteine (C, Cys), glycine (G, Gly), glutamic acid (E, Glu), glutamine (Q, Gln), histidine (H, His), isoleucine (I, Ile), leucine (L, Leu), lysine (K, Lys), methionine (M, Met), phenylalanine (F, Phe), proline (P, Pro), serine (S, Ser), threonine (T, Thr), tryptophan (W, Trp), tyrosine (Y, Tyr) and valine (V, Val).

A peptide bond, C(═O)NH, is a covalent bond formed between two amino acid molecules when the carboxyl group on one amino acid reacts with the amino group of the other amino acid in a dehydration synthesis reaction. A pentapeptide contains five (5) amino acid residues.

Dodecane peptide contains 12 amino acid residues. Tridecane peptide contains 13 amino acidresidues. Tetradecane peptide contains 14 amino acid residues. In this disclosure, a short peptide means that a peptide contains 50 or less amino acid residues. In general, a short peptide needs at least 6 amino acids residues to produce antibodies.

As used herein, the term “derivative peptide” refers to a peptide which has been modified from its original peptide, and the modification can be any chemical modification or biological modification as described herein or as known in the art.

The enzyme-linked immunosorbent assay (ELISA) is a test that uses antibodies and color change to identify a substance. The ELISA has been used as a diagnostic tool in medicine and plant pathology, as well as a quality-control check in various industries. However, the conventional ELISA usually gives inconsistent results. There are four types of ELISAs—indirect, direct, sandwich, and competitive ELISAs. In this disclosure, Biomark ELISA (modified ELISA) is used as described in the Examples.

The inventors believe that they have discovered simple, novel and unique ways for early detection and treatments of coronavirus infection as described herein.

The envelope protein can be a target for the development of early detection and treatment of coronavirus infection. For example, the enzyme-linked immunosorbent assay (ELISA) can be used for detecting antibodies against the coronavirus envelope protein. However, the antibodies produced against the full length envelop protein are usually more sensitive but less specific, and can cause cross reaction with other antigens from other viruses. One solution is to use antibodies produced against short epitope containing biopeptides or their derivatives of the envelop protein. Such antibodies produced against the short biopeptides or their derivatives can be more specific with no or reduced cross reaction.

In contrast to conventional production of antibodies from the whole envelope protein, the antibodies described herein are produced in animal or human from epitope short biopeptides or their derivatives of the envelope protein of coronavirus.

The antibodies thus produced can be used for early detection by ELISA assay on the samples (saliva or blood) taken from the human subject. The antibodies produced from whole protein are usually more sensitive but less specific, and can cross reaction with other antigens from other viruses. The antibodies produced by short biopeptides are more specific with no cross reaction.

The present application describes specific peptide sequences derived from a coronavirus envelop protein, which is necessary for the coronavirus to invade the host. The protein contains 76-109 amino acids, ranging from 8.4 to 12 kDa in size as follows: MYSFVSEETGTLIVNSVLLFLAFVVFLLVTLAILTALRLCAYCCIVNVSLVKPTVYVYSRV KNLNSSEGVPDLLV (SEQ ID NO: 1). This protein can be divided into 4 sections for antibody production in rabbits: N-Terminal, C-Terminal, and two Transmembrane sections.

According to embodiments of the application, to improve the immunogenicity and more efficiently induce an immune response, such as the production of antibodies, peptides derived from a coronavirus envelop protein are modified. The modifications can include, but are not limited to, an addition of cysteine (C) or (Cys) to the N-terminal and/or C-terminal end of the peptide sequence to facilitate binding to a carrier protein, such as a KLH, BSA, CRM197, or MBP. The modification can also include the addition of one or two alkaline lysine (K) to the N-terminal and/or C-terminal end of the peptide, to neutralize or increase pH of the peptide in solution if the peptide has too many acidic amino acid residues. According to embodiments of this application, the peptides of coronavirus envelop protein and the related peptides can be used for antibody production. In some embodiments, these peptides are the following:

N-Terminal:
(SEQ ID NO: 2)
MYSFVSEETGTLIVNS (16)
For antibody production:
(SEQ ID NO: 22)
CMYSFVSEETGTLIVNS (17)
C-Terminal:
(SEQ ID NO: 3)
RVKNLNSSEGVPDLLV (16)
For antibody production:
(SEQ ID NO: 23)
CRVKNLNSSEGVPDLLV (17)
Transmembrane 1:
(SEQ ID NO: 4)
NIVNVSLVKPTVYVYS (16)
For antibody production:
(SEQ ID NO: 24)
CNIVNVSLVKPTVYVYS (17)
Transmembrane 2:
(SEQ ID NO: 5)
FLLVTLAILTALRLC (15)
For antibody production:
(SEQ ID NO: 25)
KKFLLVTLAILTALRLC (17)

While not wishing to be bound by theory, the inventors believe that combining the N-Terminal and C-Terminal peptide can be more sensitive and more effective for the production of polyclonal antibodies:

Combining peptide:
(SEQ ID NO: 6)
MYSFVSEETGTLIVNSRVKNLNSSEGVPDLLV (32)
For antibody production:
(SEQ ID NO: 26)
CMYSFVSEETGTLIVNSRVKNLNSSEGVPDLLV (33),
or
(SEQ ID NO: 27)
CYSFVSEETGTLIVNSRVKNLNSSEGVPDLLV(32)

In addition, C-terminal peptides of Genus α, Genus β and Genus ¥ of the envelope proteins can also be used for antibodies production:

CCoV:
(SEQ ID NO: 7)
IKAYNPDEALLV (12)
For antibody production:
(SEQ ID NO: 28)
CIKAYNPDEALLV (13)
PRCV & TGEV:
(SEQ ID NO: 8)
IKAYNPDGALLV (12)
For antibody production:
(SEQ ID NO: 29)
CIKAYNPDGALLV (13)
(SEQ ID NO: 9)
IKAYNPDGDLLV (12)
For antibody production:
(SEQ ID NO: 30)
CIKAYNPDGDLLV (13)
FeCoV:
(SEQ ID NO: 10)
IKAYNPDEAFLV (12)
For antibody production:
(SEQ ID NO: 31)
CIKAYNPDEAFLV (13)
HCoV-299E:
(SEQ ID NO: 11)
HIDPFPKRVIDF (12)
For antibody production:
(SEQ ID NO: 32)
CHIDPFPKRVIDF (13)
PEDV:
(SEQ ID NO: 12)
RIDPLPSTVIDV (12)
For antibody production:
(SEQ ID NO: 33)
CRIDPLPSTVIDV (13)
HCoV-NL63:
(SEQ ID NO: 13)
QIAPVPAEVLNV (12)
For antibody production:
(SEQ ID NO: 34)
CQIAPVPAEVLNV (13)
SARS-CoV:
(SEQ ID NO: 14)
LNSSEGVPDLLV (12)
For antibody production:
(SEQ ID NO: 35)
CLNSSEGVPDLLV (13)
MERS-CoV:
(SEQ ID NO: 15)
DSKPPLPPDEWV (12)
For antibody production:
(SEQ ID NO: 36)
CDSKPPLPPDEWV (13)
HCoV-4408:
(SEQ ID NO: 16)
DVKPPVLDVDDV (12)
For antibody production:
(SEQ ID NO: 37)
CDVKPPVLDVDDV (13)
REV:
(SEQ ID NO: 17)
DEKPPVLDVDDV (12)
For antibody production:
(SEQ ID NO: 38)
CEVKPPVLDVDDV (13)
HCoV-0C43:
(SEQ ID NO: 16)
DVKPPVLDVDDV (12)
For antibody production:
(SEQ ID NO: 37)
CDVKPPVLDVDDV (13)
MHV:
(SEQ ID NO: 18)
EMRLPLLEVDDI (12)
For antibody production:
(SEQ ID NO: 39)
CEMRLPLLEVDDI (13)
HCoV-HKUl:
(SEQ ID NO: 19)
EHVIPSTLDDLI (12)
For antibody production:
(SEQ ID NO: 40)
CEHVIPSTLDDLI (13)
BatCoV:
(SEQ ID NO: 14)
LNSSEGVPDLLV (12)
For antibody production:
(SEQ ID NO: 35)
CLNSSEGVPDLLV (13)
IBV:
(SEQ ID NO: 20)
NFQDVQRDKLYS (12)
For antibody production:
(SEQ ID NO: 41)
CNFQDVQRDKLYS (13)
TCoV:
(SEQ ID NO: 21)
NEFPKNGWKNGC (12)
For antibody production:
(SEQ ID NO: 42)
CNEFPKNGWKNGC (13)

In one general aspect, the present application relates to an isolated peptide consisting of an

amino acid sequence selected from the group consisting of:

(SEQ ID NO: 2)
MYSFVSEETGTLIVNS,
(SEQ ID NO: 3)
RVKNLNSSEGVPDLLV,
(SEQ ID NO: 4)
NIVNVSLVKPTVYVYS,
(SEQ ID NO: 5)
FLLVTLAILTALRLC,
(SEQ ID NO: 6)
MYSFVSEETGTLIVNSR
VKNLNSSEGVPDLLV,
(SEQ ID NO: 7)
IKAYNPDEALLV,
(SEQ ID NO: 8)
IKAYNPDGALLV,
(SEQ ID NO: 9)
IKAYNPDGDLLV,
(SEQ ID NO: 10)
IKAYNPDEAFLV,
(SEQ ID NO: 11)
HIDPFPKRVIDF,
(SEQ ID NO: 12)
RIDPLPSTVIDV,
(SEQ ID NO: 13)
QIAPVPAEVLNV,
(SEQ ID NO: 14)
LNSSEGVPDLLV,
(SEQ ID NO: 15)
DSKPPLPPDEWV,
(SEQ ID NO: 16)
DVKPPVLDVDDV,
(SEQ ID NO: 17)
DEKPPVLDVDDV,
(SEQ ID NO: 18)
EMRLPLLEVDDI,
(SEQ ID NO: 19)
EHVIPSTLDDLI,
(SEQ ID NO: 20)
NFQDVQRDKLYS,
(SEQ ID NO: 21)
NEFPKNGWKNGC,
(SEQ ID NO: 22)
CMYSFVSEETGTLIVNS,
(SEQ ID NO: 23)
CRVKNLNSSEGVPDLLV,
(SEQ ID NO: 24)
CNIVNVSLVKPTVYVYS,
(SEQ ID NO: 25)
KKFLLVTLAILTALRLC,
(SEQ ID NO: 26)
CMYSFVSEETGTLIVNSRV
KNLNSSEGVPDLLV,
(SEQ ID NO: 27)
CYSFVSEETGTLIVNSRVK
NLNSSEGVPDLLV,
(SEQ ID NO: 28)
CIKAYNPDEALLV,
(SEQ ID NO: 29)
CIKAYNPDGALLV,
(SEQ ID NO: 30)
CIKAYNPDGDLLV,
(SEQ ID NO: 31)
CIKAYNPDEAFLV,
(SEQ ID NO: 32)
CHIDPFPKRVIDF,
(SEQ ID NO: 33)
CRIDPLPSTVIDV,
(SEQ ID NO: 34)
CQIAPVPAEVLNV,
(SEQ ID NO: 35)
CLNSSEGVPDLLV,
(SEQ ID NO: 36)
CDSKPPLPPDEWV,
(SEQ ID NO: 37)
CDVKPPVLDVDDV,
(SEQ ID NO: 38)
CEVKPPVLDVDDV,
(SEQ ID NO: 39)
CEMRLPLLEVDDI,
(SEQ ID NO: 40)
CEHVIPSTLDDLI,
(SEQ ID NO: 41)
CNFQDVQRDKLYS,
and
(SEQ ID NO: 42)
CNEFPKNGWKNGC;

or a derivative peptide thereof.

According to the embodiments of the invention, the derivative peptide can contain any modification to the disclosed peptides. For example, the modifications can include, but are not limited to, addition of cysteine (C) or (Cys) to the beginning or the end of the peptide sequence, or addition of one or two alkaline lysine (K) to the beginning or the end of the peptide sequence.

In some embodiments, the derivative peptide can contain any modification to the amino terminus and carboxy terminus:

(SEQ ID NO: 43)
R₁-MYSFVSEETGTLIVNSRVKNLNSSEGVPDLLV-R₂,

R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; R₄ is H, OH, an alkyl, aralkyl, aryl or acyl radical having up to 19 carbon atoms. A typical R₂ includes OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc and (H₃C)₂N(C═N)N(CH₃)₂. For example:

(SEQ ID NO: 44)
N-Ac-MYSFVSEETGTLIVNSRVKNLNSSEGVPDLLV-NH₂.

In some embodiments, the isolated peptide consists of an amino acid sequence selected from the group consisting of:

(SEQ ID NO: 2)
MYSFVSEETGTLIVNS,
(SEQ ID NO: 3)
RVKNLNSSEGVPDLLV,
(SEQ ID NO: 4)
NIVNVSLVKPTVYVYS,
(SEQ ID NO: 5)
FLLVTLAILTALRLC,
(SEQ ID NO: 6)
MYSFVSEETGTLIVNSR
VKNLNSSEGVPDLLV,
(SEQ ID NO: 22)
CMYSFVSEETGTLIVNS,
(SEQ ID NO: 23)
CRVKNLNSSEGVPDLLV,
(SEQ ID NO: 24)
CNIVNVSLVKPTVYVYS,
(SEQ ID NO: 25)
KKFLLVTLAILTALRLC,
(SEQ ID NO: 26)
CMYSFVSEETGTLIVNSR
VKNLNSSEGVPDLLV,
and
(SEQ ID NO: 27)
CYSFVSEETGTLIVNSRV
KNLNSSEGVPDLLV.

In some embodiments, the isolated peptide consists of an amino acid sequence selected from the group consisting of:

(SEQ ID NO: 2)
MYSFVSEETGTLIVNS,
(SEQ ID NO: 3)
RVKNLNSSEGVPDLLV,
(SEQ ID NO: 6)
MYSFVSEETGTLIVNSRVKNLNSSEGVPDLLV,
(SEQ ID NO: 22)
CMYSFVSEETGTLIVNS,
(SEQ ID NO: 23)
CRVKNLNSSEGVPDLLV,
(SEQ ID NO: 26)
CMYSFVSEETGTLIVNSRVKNLNSSEGVPDLLV,
and
(SEQ ID NO: 27)
CYSFVSEETGTLIVNSRVKNLNSSEGVPDLLV.

In some embodiments, the isolated peptide consists of an amino acid sequence selected from the group consisting of:

(SEQ ID NO: 7)
IKAYNPDEALLV,
(SEQ ID NO: 8)
IKAYNPDGALLV,
(SEQ ID NO: 9)
IKAYNPDGDLLV,
(SEQ ID NO: 10)
IKAYNPDEAFLV,
(SEQ ID NO: 11)
HIDPFPKRVIDF,
(SEQ ID NO: 12)
RIDPLPSTVIDV,
(SEQ ID NO: 13)
QIAPVPAEVLNV,
(SEQ ID NO: 14)
LNSSEGVPDLLV,
(SEQ ID NO: 15)
DSKPPLPPDEWV,
(SEQ ID NO: 16)
DVKPPVLDVDDV,
(SEQ ID NO: 17)
DEKPPVLDVDDV,
(SEQ ID NO: 18)
EMRLPLLEVDDI,
(SEQ ID NO: 19)
EHVIPSTLDDLI,
(SEQ ID NO: 20)
NFQDVQRDKLYS,
(SEQ ID NO: 21)
NEFPKNGWKNGC,
(SEQ ID NO: 28)
CIKAYNPDEALLV,
(SEQ ID NO: 29)
CIKAYNPDGALLV,
(SEQ ID NO: 30)
CIKAYNPDGDLLV,
(SEQ ID NO: 31)
CIKAYNPDEAFLV,
(SEQ ID NO: 32)
CHIDPFPKRVIDF,
(SEQ ID NO: 33)
CRIDPLPSTVIDV,
(SEQ ID NO: 34)
CQIAPVPAEVLNV,
(SEQ ID NO: 35)
CLNSSEGVPDLLV,
(SEQ ID NO: 36)
CDSKPPLPPDEWV,
(SEQ ID NO: 37)
CDVKPPVLDVDDV,
(SEQ ID NO: 38)
CEVKPPVLDVDDV,
(SEQ ID NO: 39)
CEMRLPLLEVDDI,
(SEQ ID NO: 40)
CEHVIPSTLDDLI,
(SEQ ID NO: 41)
CNFQDVQRDKLYS,
and
(SEQ ID NO: 42)
CNEFPKNGWKNGC.

Synthesis and Identification of the Peptides: The peptides can be synthesized and identified by methods described herein or as known in the art, such as synthesis by automated machine and identification by HPLC and mass spectrometry.

In another general aspect, the present application relates to a pharmaceutical composition, such as a vaccine or an immunogenic composition, which is developed from the short epitope biopeptides or their derivatives associated with the coronavirus envelop protein.

In some embodiments, the pharmaceutical composition, such as the vaccine or the immunogenic composition, comprises a peptide and a carrier protein, wherein the peptide consists of an amino acid sequence selected from the group consisting of SEQ IDs NO:2˜42 or a derivative peptide thereof.

According to the embodiments of the invention, the derivative peptide can contain any modification to the disclosed peptides. For example, the modifications can include, but are not limited to, addition of cysteine (C) or (Cys) to the beginning or the end of the peptide sequence, or addition of one or two alkaline lysine (K) to the beginning or the end of the peptide sequence.

In some embodiments, the derivative peptide can contain any modification to the amino terminus and carboxy terminus:

(SEQ ID NO: 43)
R₁-MYSFVSEETGTLIVNSRVKNLNSSEGVPDLLV-R₂,

R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; R₄ is H, OH, an alkyl, aralkyl, aryl or acyl radical having up to 19 carbon atoms. A typical R₂ includes OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc and (H₃C)₂N(C═N)N(CH₃)₂. For example:

(SEQ ID NO: 44)
N-Ac-MYSFVSEETGTLIVNSRVKNLNSSEGVPDLLV-NH₂.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or MBP, preferably KLH.

In some embodiments, the pharmaceutical composition optionally comprises another carrier other than the carrier protein. The another carrier can include one or more pharmaceutically acceptable excipients such as binders, disintegrants, swelling agents, suspending agents, emulsifying agents, wetting agents, lubricants, flavorants, sweeteners, preservatives, dyes, solubilizers and coatings.

In another general aspect, the present application relates to a method of developing antibodies against coronavirus by administering a pharmaceutical composition, such as a vaccine or an immunogenic composition, to an animal or human, wherein the pharmaceutical composition such as the vaccine or the immunogenic composition comprises a peptide and a carrier protein, and wherein the peptide consists of an amino acid sequence selected from the group consisting of SEQ IDs NO:2˜42 or a derivative peptide thereof.

In some embodiments, the derivative peptide can contain any modification to the disclosed peptides. For example, the modifications can include, but are not limited to, addition of cysteine (C) or (Cys) to the beginning or the end of the peptide sequence, or addition of one or two alkaline lysine (K) to the beginning or the end of the peptide sequence.

In some embodiments, the derivative peptide can contain any modification to the amino terminus and carboxy terminus:

(SEQ ID NO: 43)
R₁-MYSFVSEETGTLIVNSRVKNLNSSEGVPDLLV-R₂,

R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; R₄ is H, OH, an alkyl, aralkyl, aryl or acyl radical having up to 19 carbon atoms. A typical R₂ includes OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc and (H₃C)₂N(C═N)N(CH₃)₂. For example:

(SEQ ID NO: 44)
N-Ac-MYSFVSEETGTLIVNSRVKNLNSSEGVPDLLV-NH₂.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or MBP, preferably KLH.

In some embodiments, the animal is selected from the group consisting of rabbit, dog, monkey, and chimpanzee, preferably rabbit.

In some embodiments, the method comprises administering the pharmaceutical composition such as the vaccine or the immunogenic composition to a human.

In some embodiments, the antibodies are polyclonal antibodies.

In some embodiments, the pharmaceutical composition optionally comprises another carrier other than the carrier protein. The another carrier can include one or more pharmaceutically acceptable excipients such as binders, disintegrants, swelling agents, suspending agents, emulsifying agents, wetting agents, lubricants, flavorants, sweeteners, preservatives, dyes, solubilizers and coatings.

According to the embodiments of the invention, the pharmaceutical composition can be administered by any methods known in the art. These administration methods include, but are not limited to, subcutaneous route, intramuscular route, intradermal, or intranasal route. In preferred embodiments, the pharmaceutical composition is administered subcutaneously.

In another general aspect, the present application relates to an antibody against coronavirus, wherein the antibody is developed by the methods of the invention.

In some embodiments, the antibody is developed by a method comprising administering a pharmaceutical composition, such as a vaccine or an immunogenic composition, to an animal or human, wherein the pharmaceutical composition comprises a peptide and a carrier protein, and wherein the peptide consists of an amino acid sequence selected from the group consisting of SEQ IDs NO:2˜42 or a derivative peptide thereof.

In some embodiments, the derivative peptide can contain any modification to the disclosed peptides. For example, the modifications can include, but are not limited to, addition of cysteine (C) or (Cys) to the beginning or the end of the peptide sequence, or addition of one or two alkaline lysine (K) to the beginning or the end of the peptide sequence.

In some embodiments, the derivative peptide can contain any modification to the amino terminus and carboxy terminus:

(SEQ ID NO: 43)
R₁-MYSFVSEETGTLIVNSRVKNLNSSEGVPDLLV-R₂,

R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; R₄ is H, OH, an alkyl, aralkyl, aryl or acyl radical having up to 19 carbon atoms. A typical R₂ includes OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc and (H₃C)₂N(C═N)N(CH₃)₂. For example:

(SEQ ID NO: 44)
N-Ac-MYSFVSEETGTLIVNSRVKNLNSSEGVPDLLV-NH₂.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or MBP, preferably KLH.

In some embodiments, the animal is selected from the group consisting of rabbit, dog, monkey, and chimpanzee, preferably rabbit.

In some embodiments, the method comprises administering the pharmaceutical composition to a human.

In some embodiments, the antibody is a polyclonal antibody.

In some embodiments, the pharmaceutical composition optionally comprises another carrier other than the carrier protein. The another carrier can include one or more pharmaceutically acceptable excipients such as binders, disintegrants, swelling agents, suspending agents, emulsifying agents, wetting agents, lubricants, flavorants, sweeteners, preservatives, dyes, solubilizers and coatings.

In another general aspect, the present invention relates to a method of detecting coronavirus in a subject in need thereof, the method comprising:
c. obtaining a sample from the subject; and
d. detecting in the sample the presence of one or more antibodies targeting a peptide, or the presence of one or more antigens that bind specifically to the antibody disclosed in the application, wherein the peptide consists of an amino acid sequence selected from the group consisting of SEQ IDs NO:2˜42 or a derivative peptide thereof.

In some embodiments, the derivative peptide can contain any modification to the disclosed peptides. For example, the modifications can include, but are not limited to, addition of cysteine (C) or (Cys) to the beginning or the end of the peptide sequence, or addition of one or two alkaline lysine (K) to the beginning or the end of the peptide sequence.

In some embodiments, the derivative peptide can contain any modification to the amino terminus and carboxy terminus:

(SEQ ID NO: 43)
R₁-MYSFVSEETGTLIVNSRVKNLNSSEGVPDLLV-R₂,

R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; R₄ is H, OH, an alkyl, aralkyl, aryl or acyl radical having up to 19 carbon atoms. A typical R₂ includes OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc and (H₃C)₂N(C═N)N(CH₃)₂. For example:

(SEQ ID NO: 44)
N-Ac-MYSFVSEETGTLIVNSRVKNLNSSEGVPDLLV-NH₂.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the sample can be any biological sample from the subject, such as saliva, blood, tissue and urine. In certain embodiments, the sample is saliva, nasal swab, or serum.

In some embodiments, the antibodies is polyclonal antibodies.

In some embodiments, the antibodies are produced in animal, preferably selected from the group consisting of rabbit, dog, monkey, and chimpanzee, more preferably rabbit.

In some embodiments, the antibodies are produced in human.

In some embodiments, the antibodies can be detected by any methods described herein or as known in the art, e.g., immunoprecipitation assays such as enzyme-linked immunosorbent assay (ELISA), immunoblotting such as dot blot technique, and immunosorbent assays.

In some embodiments, the ELISA is direct ELISA, indirect ELISA, sandwich ELISA, or competitive ELISA.

In some embodiments, the subject has no symptom of coronavirus infection at the time of the detection of the coronavirus.

In some embodiments, the coronavirus is selected from the group consisting of SARS-CoV-2, SARS virus, MERS virus, and common cold virus.

In another general aspect, the present application relates to a method of treating a coronavirus infection in a subject in need thereof, the method comprising administering to the subject a pharmaceutical composition, such as a vaccine or an immunogenic composition, comprising a peptide and a carrier protein, and wherein the peptide consists of an amino acid sequence selected from the group consisting of SEQ IDs NO:2˜42 or a derivative peptide thereof.

In some embodiments, the derivative peptide can contain any modification to the disclosed peptides. For example, the modifications can include, but are not limited to, addition of cysteine (C) or (Cys) to the beginning or the end of the peptide sequence, or addition of one or two alkaline lysine (K) to the beginning or the end of the peptide sequence.

In some embodiments, the derivative peptide can contain any modification to the amino terminus and carboxy terminus:

(SEQ ID NO: 43)
R₁-MYSFVSEETGTLIVNSRVKNLNSSEGVPDLLV-R₂,

R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; R₄ is H, OH, an alkyl, aralkyl, aryl or acyl radical having up to 19 carbon atoms. A typical R₂ includes OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc and (H₃C)₂N(C═N)N(CH₃)₂. For example:

(SEQ ID NO: 44)
N-Ac-MYSFVSEETGTLIVNSRVKNLNSSEGVPDLLV-NH₂.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or MBP, preferably KLH.

In some embodiments, the subject has no symptom of coronavirus infection at the time of the treatment of the coronavirus.

In some embodiments, the coronavirus is selected from the group consisting of SARS-CoV-2, SARS virus, MERS virus, and common cold virus.

In some embodiments, the pharmaceutical composition optionally comprises another carrier other than the carrier protein. The another carrier can include one or more pharmaceutically acceptable excipients such as binders, disintegrants, swelling agents, suspending agents, emulsifying agents, wetting agents, lubricants, flavorants, sweeteners, preservatives, dyes, solubilizers and coatings.

According to the embodiments of the invention, the pharmaceutical composition can be administered by any methods known in the art. These administration methods include, but are not limited to, subcutaneous route, intramuscular route, intradermal, or intranasal route. In preferred embodiments, the pharmaceutical composition is administered subcutaneously.

In another general aspect, the present application relates to a method of treating a coronavirus infection in a subject in need thereof, the method comprising administering to the subject an antibody according to embodiments of the application.

In some embodiments, the antibody is developed by a method comprising administering a pharmaceutical composition, such as a vaccine or an immunogenic composition, to an animal or human, wherein the pharmaceutical composition comprises a peptide and a carrier protein, and wherein the peptide consists of an amino acid sequence selected from the group consisting of SEQ IDs NO:2˜42 or a derivative peptide thereof.

In some embodiments, the derivative peptide can contain any modification to the amino terminus and carboxy terminus:

(SEQ ID NO: 43)
R₁-MYSFVSEETGTLIVNSRVKNLNSSEGVPDLLV-R₂,

R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; R₄ is H, OH, an alkyl, aralkyl, aryl or acyl radical having up to 19 carbon atoms. A typical R₂ includes OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc and (H₃C)₂N(C═N)N(CH₃)₂. For example:

(SEQ ID NO: 44)
N-Ac-MYSFVSEETGTLIVNSRVKNLNSSEGVPDLLV-NH₂.

In some embodiments, the derivative peptide can contain any modification to the disclosed peptides. For example, the modifications can include, but are not limited to, addition of cysteine (C) or (Cys) to the beginning or the end of the peptide sequence, or addition of one or two alkaline lysine (K) to the beginning or the end of the peptide sequence.

In some embodiments, the peptide consists of an amino acid sequence selected from the group consisting of SEQ IDs NO:2˜6 and SEQ IDs NO:22˜27.

In some embodiments, the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

In some embodiments, the peptide consists of an amino acid sequence selected from the group consisting of SEQ IDs NO:7˜21 and SEQ IDs NO:28˜42.

In some embodiments, the carrier protein is keyhole limpet hemocyanin (KLH), bovine serum albumin (BSA), CRM197, or MBP, preferably KLH.

In some embodiments, the subject has no symptom of coronavirus infection at the time of the treatment of the coronavirus.

In some embodiments, the coronavirus is selected from the group consisting of SARS-CoV-2, SARS virus, MERS virus, and common cold virus.

In some embodiments, the pharmaceutical composition optionally comprises another carrier other than the carrier protein. The another carrier can include one or more pharmaceutically acceptable excipients such as binders, disintegrants, swelling agents, suspending agents, emulsifying agents, wetting agents, lubricants, flavorants, sweeteners, preservatives, dyes, solubilizers and coatings.

According to the embodiments of the invention, the antibodies can be administered by any methods known in the art. These administration methods include, but are not limited to, subcutaneous route, intramuscular route, intradermal, or intranasal route.

In preferred embodiments, the antibodies are administered subcutaneously.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the following Examples and appended claims.

### EMBODIMENTS

The present application provides also the following non-limiting embodiments.

Embodiment 1 is an immunogenic composition comprising a peptide and a carrier protein, wherein the peptide consists of an amino acid sequence selected from the group consisting of SEQ IDs NO:2˜42 or a derivative peptide thereof.

Embodiment 1a is the immunogenic composition of embodiment 1, wherein the peptide consists of an amino acid sequence selected from the group consisting of SEQ IDs NO:2˜6 and SEQ IDs NO:22˜27.

Embodiment 1b is the immunogenic composition of embodiment 1, wherein the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

Embodiment 1c is the immunogenic composition of embodiment 1, wherein the peptide consists of an amino acid sequence selected from the group consisting of SEQ IDs NO:7˜21 and SEQ IDs NO:28˜42.

Embodiment 1d is the immunogenic composition of any one of embodiments 1-1c, wherein the carrier protein is keyhole limpet hemocyanin (KLH), bovine serum albumin (BSA), CRM197, or maltose binding protein (MBP), preferably KLH.

Embodiment 2 is a method of developing antibodies against coronavirus, the method comprising administering an immunogenic composition to an animal or human, wherein the immunogenic composition comprises a peptide and a carrier protein, wherein the peptide consists of an amino acid sequence selected from the group consisting of SEQ IDs NO:2˜42 or a derivative peptide thereof.

Embodiment 2a is the method of embodiment 2, wherein the peptide consists of an amino acid sequence selected from the group consisting of SEQ IDs NO:2˜6 and SEQ IDs NO:22˜27.

Embodiment 2b is the method of embodiment 2, wherein the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

Embodiment 2c is the method of embodiment 2, wherein the peptide consists of an amino acid sequence selected from the group consisting of SEQ IDs NO:7˜21 and SEQ IDs NO:28˜42.

Embodiment 2d is the method of any one of embodiments 2-2c, wherein the carrier protein is keyhole limpet hemocyanin (KLH), bovine serum albumin (BSA), CRM197, or maltose binding protein (MBP), preferably KLH.

Embodiment 3 is an antibody developed by the method of any one of embodiments 2-2d.

Embodiment 3a is the antibody of embodiment 3, wherein the peptide consists of an amino acid sequence selected from the group consisting of SEQ IDs NO:2˜6 and SEQ IDs NO:22˜27.

Embodiment 3b is the antibody of embodiment 3, wherein the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

Embodiment 3c is the antibody of embodiment 3, wherein the peptide consists of an amino acid sequence selected from the group consisting of SEQ IDs NO:7˜21 and SEQ IDs NO:28˜42.

Embodiment 3d is the antibody of any one of embodiments 3-3c, wherein the antibody is a polyclonal antibody.

Embodiment 4 is a method of detecting coronavirus in a subject in need thereof, the method comprising:
a. obtaining a sample from the subject; and
b. detecting in the sample the presence of one or more antibodies targeting a peptide, or the presence of one or more antigens that bind specifically to the antibody of any one of embodiments 3-3d, wherein the peptide consists of an amino acid sequence selected from the group consisting of SEQ IDs NO:2˜42 or a derivative peptide thereof.

Embodiment 4a is the method of embodiment 4, wherein the peptide consists of an amino acid sequence selected from the group consisting of SEQ IDs NO:2˜6 and SEQ IDs NO:22˜27.

Embodiment 4b is the method of embodiment 4, wherein the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

Embodiment 4c is the method of embodiment 4, wherein the peptide consists of an amino acid sequence selected from the group consisting of SEQ IDs NO:7˜21 and SEQ IDs NO:28˜42.

Embodiment 4d is the method of any one of embodiments 4-4c, wherein the sample is saliva.

Embodiment 4e is the method of embodiment 4, wherein the one or more antibodies or antigens are detected by an enzyme-linked immunosorbent assay (ELISA).

Embodiment 4f is the method of embodiment 4, wherein the subject has no symptom of coronavirus infection at the time of the detection of the coronavirus.

Embodiment 4f is the method of embodiment 4, wherein the coronavirus is selected from the group consisting of SARS-CoV-2, SARS virus, MERS virus, and common cold virus.

Embodiment 5 is a method of treating a coronavirus infection in a subject in need thereof, the method comprising administering to the subject a pharmaceutical composition, such as a vaccine or an immunogenic composition, comprising a peptide and a carrier protein, wherein the peptide consists of an amino acid sequence selected from the group consisting of SEQ IDs NO:2˜42 or a derivative peptide thereof.

Embodiment 5a is the method of embodiment 5, wherein the peptide consists of an amino acid sequence selected from the group consisting of SEQ IDs NO:2˜6 and SEQ IDs NO:22˜27.

Embodiment 5b is the method of embodiment 5, wherein the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

Embodiment 5c is the method of embodiment 5, wherein the peptide consists of an amino acid sequence selected from the group consisting of SEQ IDs NO:7˜21 and SEQ IDs NO:28˜42.

Embodiment 5d is the method of any one of embodiments 5-5c, wherein the carrier protein is keyhole limpet hemocyanin (KLH), bovine serum albumin (BSA), CRM197, or maltose binding protein (MBP), preferably KLH.

Embodiment 5e is the method of any one of embodiments 5-5d, wherein the subject has no symptom of coronavirus infection at the time of the treatment of the coronavirus.

Embodiment 5f is the method of any one of embodiments 5-5e, where the coronavirus is selected from the group consisting of SARS-CoV-2, SARS virus, MERS virus, and common cold virus.

Embodiment 6 is a method of treating a coronavirus infection in a subject in need thereof, the method comprising administering to the subject an antibody, wherein the antibody is developed by a method comprising administering a pharmaceutical composition, such as a vaccine or an immunogenic composition, to an animal or human, wherein the pharmaceutical composition comprises a peptide and a carrier protein, and wherein the peptide consists of an amino acid sequence selected from the group consisting of SEQ IDs NO:2˜42 or a derivative peptide thereof.

Embodiment 6a is the method of embodiment 6, wherein the peptide consists of an amino acid sequence selected from the group consisting of SEQ IDs NO:2˜6 and SEQ IDs NO:22˜27.

Embodiment 6b is the method of embodiment 6, wherein the peptide consists of an amino acid sequence selected from the group consisting of SEQ ID NO:2, SEQ ID NO:3, SEQ ID NO:6, SEQ ID NO:22, SEQ ID NO:23, SEQ ID NO:26, and SEQ ID NO:27.

Embodiment 6c is the method of embodiment 6, wherein the peptide consists of an amino acid sequence selected from the group consisting of SEQ IDs NO:7˜21 and SEQ IDs NO:28˜42.

Embodiment 6d is the method of any one of embodiments 6-6c, wherein the carrier protein is keyhole limpet hemocyanin (KLH), bovine serum albumin (BSA), CRM197, or maltose binding protein (MBP), preferably KLH.

Embodiment 6e is the method of any one of embodiments 6-6d, wherein the subject has no symptom of coronavirus infection at the time of the treatment of the coronavirus.

Embodiment 6f is the method of any one of embodiments 6-6e, where the coronavirus is selected from the group consisting of SARS-CoV-2, SARS virus, MERS virus, and common cold virus.

### EXAMPLES

The following examples of the invention are to further illustrate the nature of the invention. It should be understood that the following examples do not limit the invention and the scope of the invention is to be determined by the appended claims.

### Example 1: Rabbit Polyclonal Antibody Production (Project AP43690)

Materials: Four peptides of SEQ ID NOs:22-25 were used for the rabbit polyclonal antibody production:

(SEQ ID NO: 22)
CMYSFVSEETGTLIVNS
(SEQ ID NO: 23)
CRVKNLNSSEGVPDLLV
(SEQ ID NO: 24)
CNIVNVSLVKPTVYVYS
(SEQ ID NO: 25)
KKFLLVTLAILTALRLC

Methods: For the rabbit polyclonal antibody production, each peptide antigen was emulsified in Complete Freund's Adjuvant (CFA) which contained keyhole limpet hemocyanin (KLH) forinitial subcutaneous injections (S.C.). Incomplete Freund's Adjuvant (IFA) was used for subsequent boost injections. Peptide-KLH conjugate was formed due to the presence of Cys at the N- or C-terminus of the peptide. Two to four rabbits were used for each peptide at the amount of 3.0 mg peptide per rabbit. The production procedure is listed in Table 1.

TABLE 1
Rabbit Polyclonal Antibody Production Procedure

| | | |
|---|---|---|
| Primary | Day 1 | 0.5 mg peptide-KLH conjugate per |
| immunization | | rabbit, S.C. |
| 1st boost | Day 14 | 0.5 mg peptide-KLH conjugate per |
| | | rabbit, S.C. |
| Test bleed and | Day 24 | 100 μl serum per rabbit; ELISA and |
| ELISA | | immune response evaluation. |
| 2nd boost | Day 35 | 0.5 mg peptide-KLH conjugate per |
| | | rabbit, S.C. |
| Production bleed | Day 45 | ELISA test |
| 3rd boost | Day 49 | 0.5 mg peptide-KLH conjugate per |
| | | rabbit, S.C. |
| Production bleed | Day 59 | ELISA test |
| 4th boost | Day 63 | 0.5 mg peptide-KLH conjugate per |
| | | rabbit, S.C. |
| Production bleed | Day 73 | ELISA test |
| 5th boost | Day 77 | 0.5 mg peptide-KLH conjugate per |
| | | rabbit, S.C. |
| Production bleed | Day 87 | 40 ml serum per rabbit |

Results: After the rabbit polyclonal antibodies were produced and purified, each antibody was analyzed by HPLC and mass chromatography. The production results are listed in Table 2 and Table 3 below.

TABLE 2
Rabbit ID# and Purified Antibody Concentration

| Rabbit ID # | Concentration of Purified Antibody |
|---|---|
| E14826 | 1.99 mg/ml |
| E14827 | 2.15 mg/ml |
| E14828 | 2.38 mg/ml |
| E14829 | 1.80 mg/ml |
| E14830 | 2.40 mg/ml |
| E14831 | 2.30 mg/ml |
| E14832 | 1.00 mg/ml |
| E14833 | 0.45 mg/ml |

TABLE 3
Quantity of Peptides and Antibodies

| | | | | No. of |
|---|---|---|---|---|
| Description | Lot # | Quantity | | Vials |
| Naked peptide | KKFLLVTLAILTALRLC (SEQ ID NO: 25) | 20 | mg | 1 |
| Naked peptide | CNIVNVSLVKPTVYVYS (SEQ ID NO: 24) | 20 | mg | 1 |
| Naked peptide | CRVKNLNSSEGVPDLLV (SEQ ID NO: 23) | 20 | mg | 1 |
| Naked peptide | CMYSFVSEETGTLIVNS (SEQ ID NO: 22) | 20 | mg | 1 |
| Primary immunization, | E14826, E14827, E14828, E14829, E14830, | 100 | μl | 8 |
| antiserum | E14831, E14832, E14833 | | | |
| Small scale antibody | E14826, E14827, E14828, E14829, E14830, | 100 | μl | 8 |
| | E14831, E14832, E14833 | | | |
| Large scale antibody | E14833 | 700 | μl | 2 |
| Large scale antibody | E14827, E14832 | 1000 | μl | 2 |
| Large scale antibody | E14826, E14828, E14830, E14831 | 900 | μl | 4 |
| Large scale antibody | E14829 | 1200 | μl | 1 |

### Example 2: Rabbit Polyclonal Antibody Production (Project AP43900)

Materials: In this Example, the peptide of SEQ ID NO: 27 was used for polyclonal antibody production in rabbits:

(32)
(SEQ ID NO: 27)
CYSFVSEETGTLIVNSRVKNLNSSEGVPDLLV

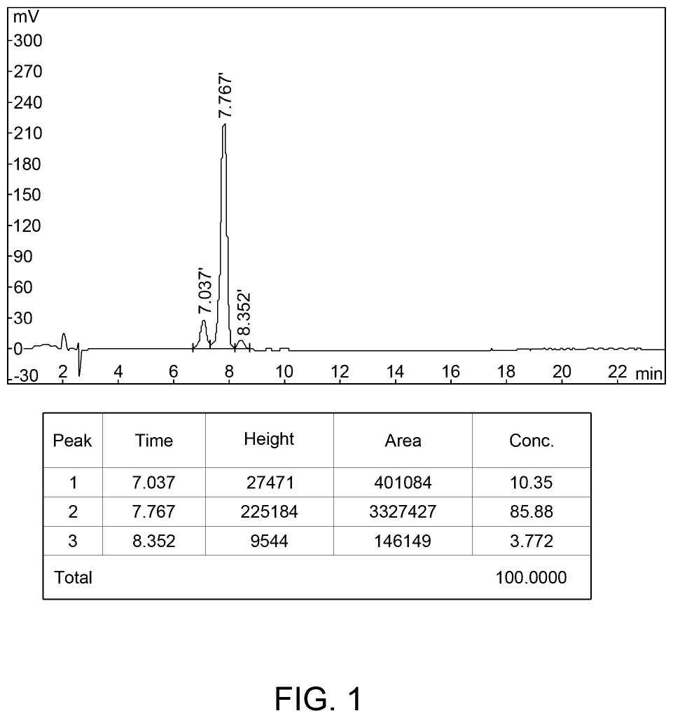

The peptide of SEQ ID NO: 27 was synthesized by ABclonal Technology (Woburn, MA), which was obtained at 59 mg with ≥85% purity. The HPLC of this peptide is shown in FIG. 1. The HPLC condition is the following:

| Column: | 4.6 × 250 mm, SinoChrom ODDS-BP | |
|---|---|---|
| Solvent A: | A: 0.1% Trifluoroacetic acid in 100% Acetonitrile | |
| Solvent B: | B: 0.1% Trifluoroacetic acid in 100% Water | |
| | Gradient: | |
| | A | B |
| 0.0 min | 25% | 75% |
| 25.0 min | 50% | 50% |
| 25.1 min | 100% | 0% |
| 30.0 min | Stop | |

| | |
|---|---|
| Volume: | 5 μl |
| Wavelength: | 220 nm |
| Flow Rate: | 1.0 ml/min |

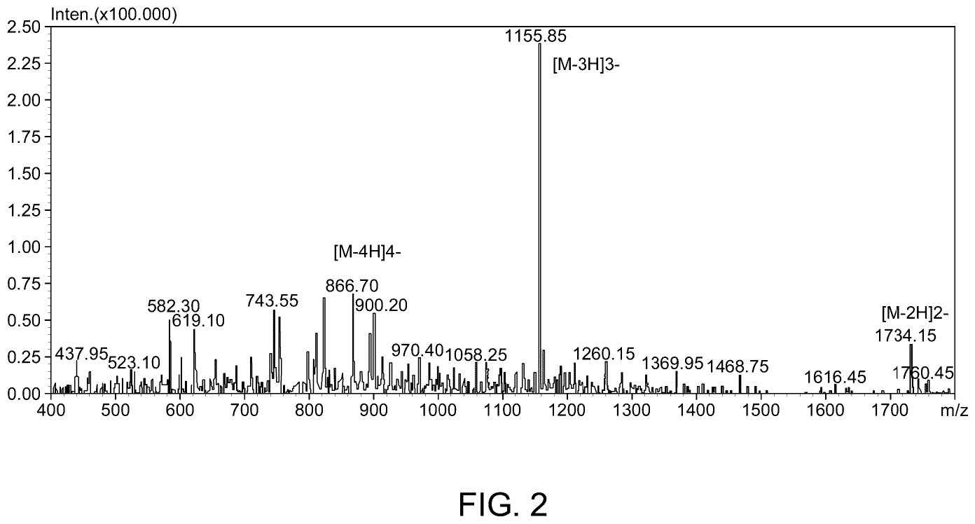

The mass spectrum at FIG. 2 indicates that the molecular weight (MW) of this peptide is 3470.92.

Methods: The polyclonal antibody production in rabbits was conducted at ABclonal Technology (Woburn, MA), following the procedure described in Table 4. For the rabbit polyclonal antibody production, the peptide antigen of SEQ ID NO: 27 was emulsified in Complete Freund's Adjuvant (CFA) which contained keyhole limpet hemocyanin (KLH) for the first immunization. Incomplete Freund's Adjuvant (IFA) was used for subsequent immunizations. Four New Zealand rabbits were used for the production.

TABLE 4
Rabbit Polyclonal Antibody Production Procedure

| | | |
|---|---|---|
| 1^{st} immunization | Day 1 | 0.7 mg peptide-KLH conjugate per rabbit |
| 2^{nd} immunization | Day 12 | 0.35 mg peptide-KLH conjugate per rabbit |
| 3^{rd} immunization | Day 26 | 0.35 mg peptide-KLH conjugate per rabbit |
| 4^{th} immunization | Day 40 | 0.35 mg peptide-KLH conjugate per rabbit |
| 5^{th} immunization | Day 54 | 0.35 mg peptide-KLH conjugate per rabbit |
| Blood collection | Day 66 | |

Results: The produced antibody was purified by antibody antigen affinity chromatography. The production results are listed in Table 5 and Table 6 below.

TABLE 5
Rabbit ID# and Purified Antibody Concentration

| Rabbit ID # | Concentration of Purified Antibody |
|---|---|
| E17042 | 1.33 mg/ml |
| E17043 | 1.05 mg/ml |
| E17044 | 1.92 mg/ml |
| E17045 | 1.62 mg/ml |

TABLE 6
Quantity of Peptides and Antibodies

| | | | No. of |
|---|---|---|---|
| Description | Lot # | Quantity | Vials |
| Naked peptide | CYSFVSEETGTLIVNSRVKNLNSSEGVPDLLV | 40 mg | 1 |
| | (32) (SEQ ID NO: 27) | | |
| Primary immunization, | E17042, E17043, E17044, E17045 | 100 μl | 4 |
| antiserum | | | |
| Large scale antibody | E17042 | 800 μl | 2 |
| Large scale antibody | E17044 | 1100 μl | 1 |
| Large scale antibody | E17043 | 850 μl | 2 |
| Large scale antibody | E17045 | 1200 μl | 1 |

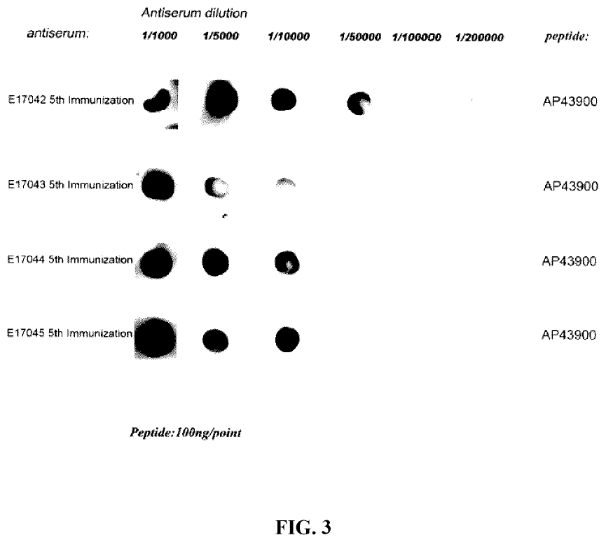

FIG. 3 shows the dot blot testing data of purified antibodies, which indicates that post 5^{th} immunization antisera from 5 rabbits were positive against the antigen peptide of SEQ ID NO: 27.

### Example 3: Rabbit Polyclonal Antibody Production (Project AP43709)

Materials: Sixteen peptide were used for polyclonal antibody production in rabbits:

(SEQ ID NO: 28)
CIKAYNPDEALLV
(SEQ ID NO: 29)
CIKAYNPDGALLV
(SEQ ID NO: 30)
CIKAYNPDGDLLV
(SEQ ID NO: 31)
CIKAYNPDEAFLV
(SEQ ID NO: 32)
CHIDPFPKRVIDF
(SEQ ID NO: 33)
CRIDPLPSTVIDV
(SEQ ID NO: 34)
CQIAPVPAEVLNV
(SEQ ID NO: 35)
CLNSSEGVPDLLV
(SEQ ID NO: 36)
CDSKPPLPPDEWV
(SEQ ID NO: 37)
CDVKPPVLDVDDV
(SEQ ID NO: 38)
CEVKPPVLDVDDV
(SEQ ID NO: 37)
CDVKPPVLDVDDV
(SEQ ID NO: 39)
CEMRLPLLEVDDI
(SEQ ID NO: 40)
CEHVIPSTLDDLI
(SEQ ID NO: 41)
CNFQDVQRDKLYS
(SEQ ID NO: 21)
NEFPKNGWKNGC

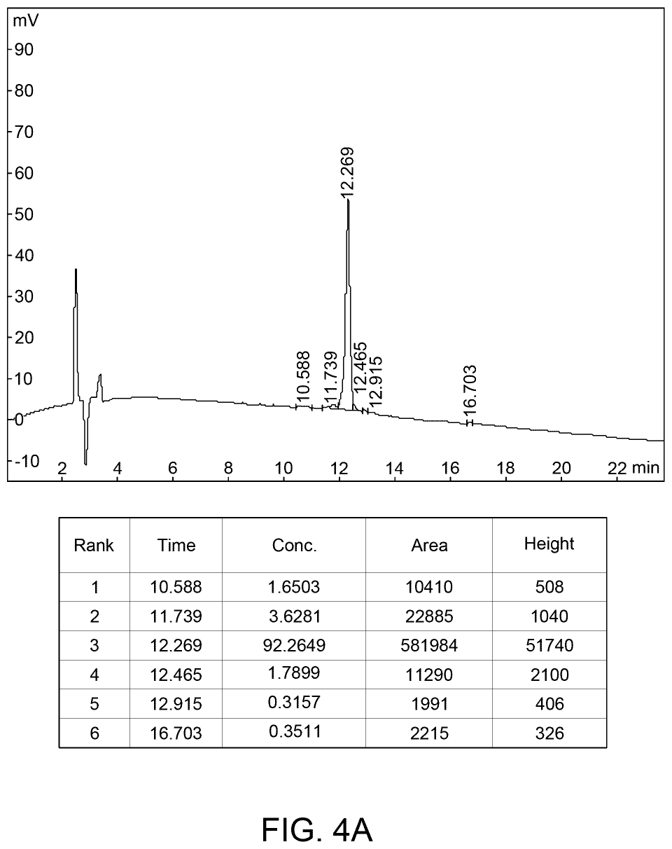

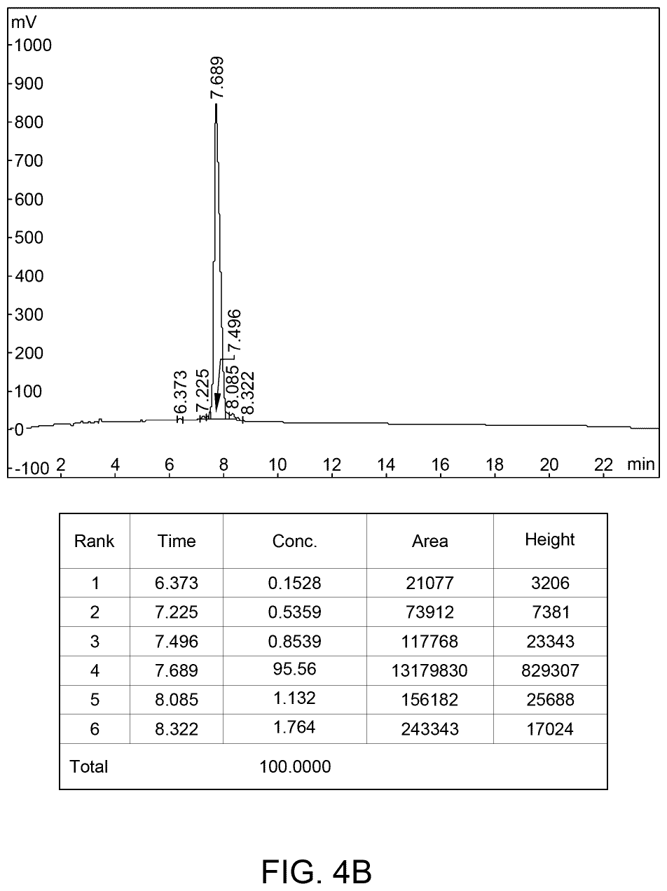

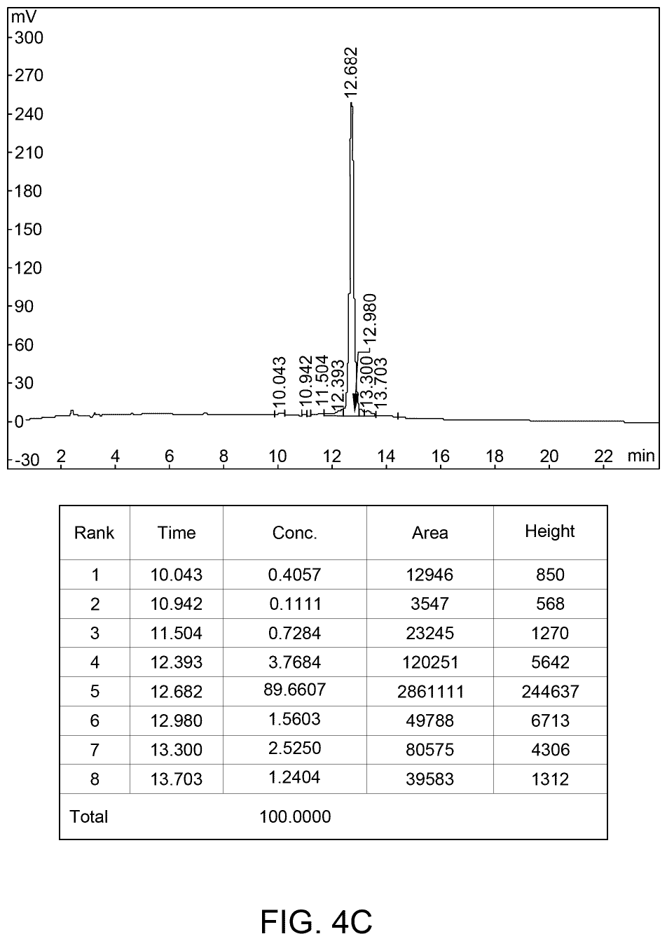

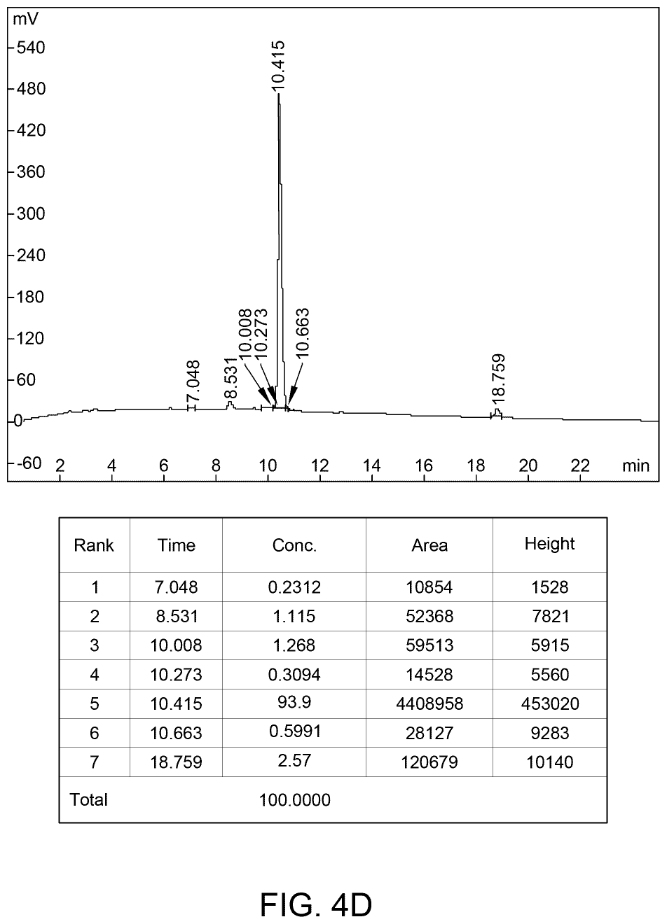

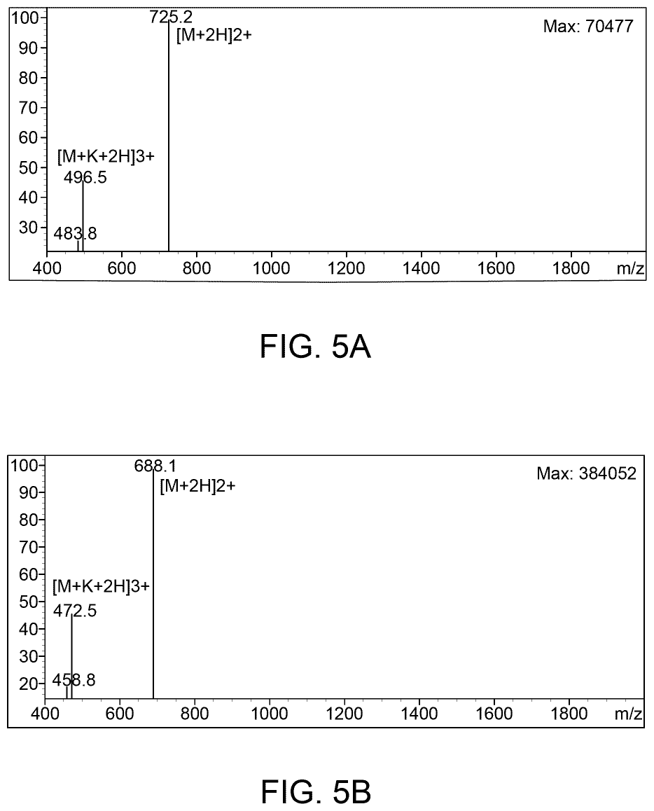

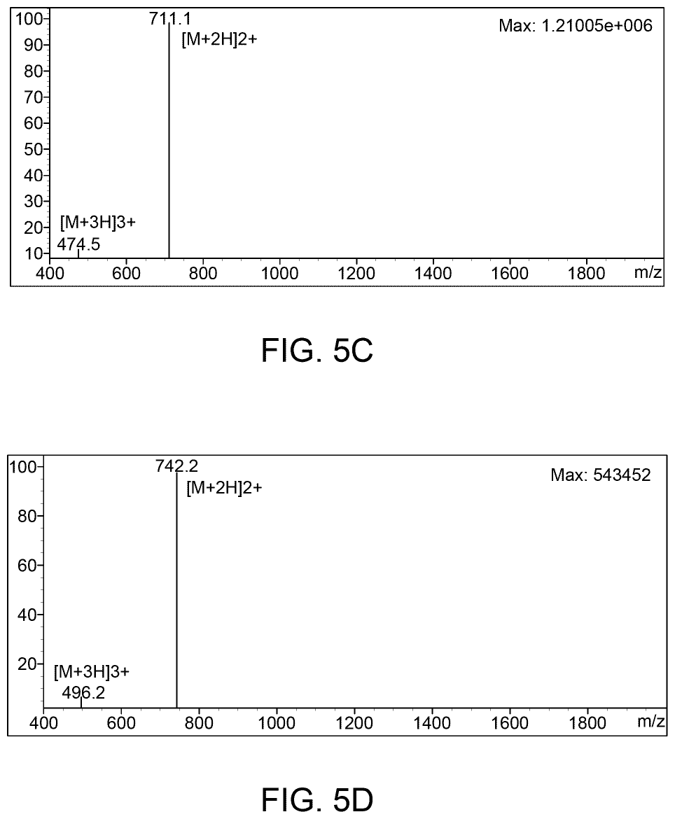

All 16 peptide were also synthesized by ABclonal Technology (Woburn, MA). These peptides were characterized by HPLC and mass spectrometry. The HPLC of some peptides (SEQ IDs NO:28-31) are shown in FIGS. 4A-D. The corresponding mass spectra of these peptides (SEQ IDs NO:28-31) are shown in FIGS. 5A-D.

Methods: The polyclonal antibody production in rabbits was conducted at ABclonal Technology (Woburn, MA), following the same procedure as described in Table 4 above. Two New Zealand rabbits were used for each peptide.

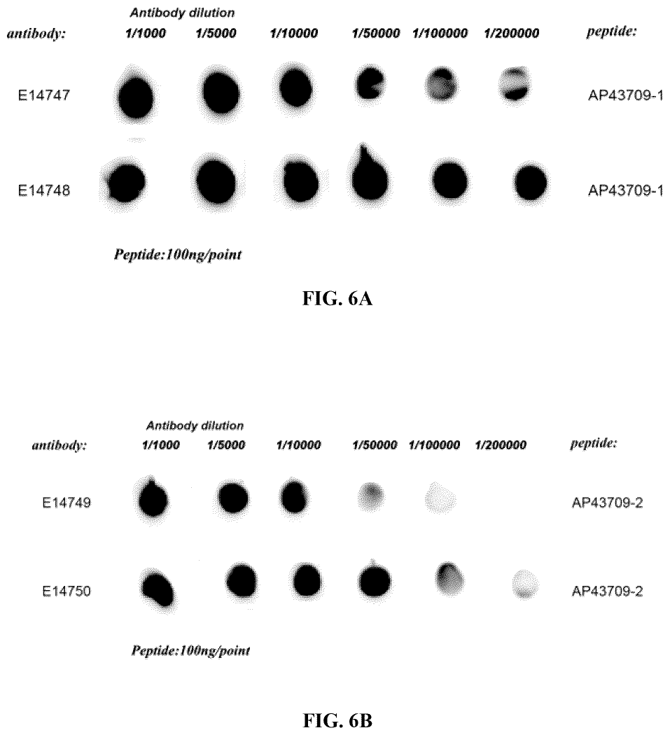

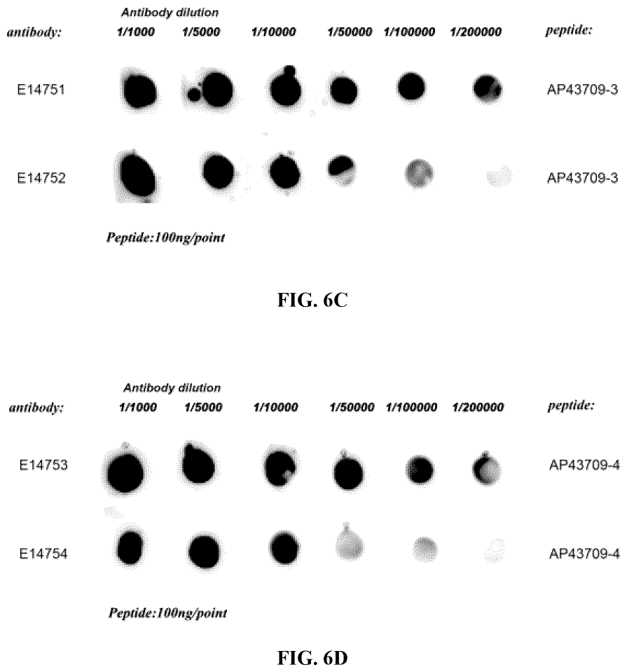

Results: The produced antibodies were purified by antibody antigen affinity chromatography. For all the 16 peptides, the dot blot testing data of purified antibodies indicated that post 5^{th} immunization antisera from the rabbits were positive against the peptide. The dot blot testing of some peptides (SEQ IDs NO:28-31) are shown in FIGS. 6A-D.

The production results are listed in Table 7 and Table 8 below.

TABLE 5
Rabbit ID# and Purified Antibody Concentration

| Sequence | Rabbit | Concentration of Purified |
|---|---|---|
| ID NO | ID # | Antibody |
| 30 | E14747 and E14748 | 2.78 mg/ml and 2.03 mg/ml |
| 31 | E14749 and E14750 | 1.36 mg/ml and 1.37 mg/ml |
| 32 | E14751 and E14752 | 2.57 mg/ml and 2.47 mg/ml |
| 33 | E14753 and E14754 | 2.77 mg/ml and 1.80 mg/ml |
| 34 | E14755 and E14756 | 2.41 mg/ml and 1.14 mg/ml |
| 35 | E14757 and E14758 | 2.73 mg/ml and 2.04 mg/ml |
| 36 | E14838 and E14839 | 1.58 mg/ml and 1.41 mg/ml |
| 37 | E14840 and E14841 | 1.31 mg/ml and 2.70 mg/ml |
| 38 | E14842 and E14843 | 2.05 mg/ml and 2.67 mg/ml |
| 39 | E14844 and E14845 | 2.69 mg/ml and 2.36 mg/ml |
| 40 | E14846 and E14847 | 2.84 mg/ml and 1.19 mg/ml |
| 41 | E14848 and E14849 | 2.38 mg/ml and 1.55 mg/ml |
| 42 | E14850 and E14851 | 2.84 mg/ml and 1.78 mg/ml |
| 43 | E14852 and E14853 | 2.73 mg/ml and 2.12 mg/ml |
| 45 | E14854 and E14855 | 1.70 mg/ml and 2.08 mg/ml |
| 23 | E14856 and E14857 | 1.88 mg/ml and 0.85 mg/ml |

TABLE 8
Quantity of Peptides and Antibodies

| | | | No. of |
|---|---|---|---|
| Description | lot # | Quantity | vials |
| Naked peptide | CIKAYNPDEALLV (SEQ ID NO: 28) | 20 mg | 1 |
| Pre-imm. antiserum, Rabbit | E14747, E14748 | 100 μL | 2 |
| Small scale antibody, | E14747, E14748 | 100 μL | 2 |
| Rabbit | | | |
| Large scale antibody, | E14747 | 750 μL | 1 |
| Rabbit | | | |
| Large scale antibody, | E14748 | 700 μL | 1 |
| Rabbit | | | |
| Naked peptide | CIKAYNPDGALLV (SEQ ID NO: 29) | 20 mg | 1 |
| Pre-imm. antiserum, Rabbit | E14749, E14750 | 100 μL | 2 |
| Small scale antibody, | E14749, E14750 | 100 μL | 2 |
| Rabbit | | | |
| Large scale antibody, | E14750 | 900 μL | 1 |
| Rabbit | | | |
| Large scale antibody, | E14749 | 750 μL | 2 |
| Rabbit | | | |
| Naked peptide | CIKAYNPDGDLLV (SEQ ID NO: 30) | 20 mg | 1 |
| Pre-imm. antiserum, Rabbit | E14751, E14752 | 100 μL | 2 |
| Small scale antibody, | E14751, E14752 | 100 μL | 2 |
| Rabbit | | | |
| Large scale antibody, | E14752 | 900 μL | 1 |
| Rabbit | | | |
| Large scale antibody, | E14751 | 800 μL | 1 |
| Rabbit | | | |
| Naked peptide | CIKAYNPDEAFLV (SEQ ID NO: 31) | 20 mg | 1 |
| Pre-imm. antiserum, Rabbit | E14753, E14754 | 100 μL | 2 |
| Small scale antibody, | E14753, E14754 | 100 μL | 2 |
| Rabbit | | | |
| Large scale antibody, | E14754 | 900 μL | 1 |
| Rabbit | | | |
| Large scale antibody, | E14753 | 700 μL | 1 |
| Rabbit | | | |
| Naked peptide | CHIDPFPKRVIDF (SEQ ID NO: 32) | 20 mg | 1 |
| Pre-imm. antiserum, Rabbit | E14834, E14835 | 100 μL | 2 |
| Small scale antibody, | E14834, E14835 | 100 μL | 2 |
| Rabbit | | | |
| Large scale antibody, | E14834 | 900 μL | 1 |
| Rabbit | | | |
| Naked peptide | CRIDPLPSTVIDV (SEQ ID NO: 33) | 20 mg | 1 |
| Pre-imm. antiserum, Rabbit | E14836, E14837 | 100 μL | 2 |
| Small scale antibody, | E14836, E14837 | 100 μL | 2 |
| Rabbit | | | |
| Large scale antibody, | E14837 | 1000 μL | 1 |
| Rabbit | | | |
| Large scale antibody, | E14836 | 800 μL | 1 |
| Rabbit | | | |
| Naked peptide | CQIAPVPAEVLNV (SEQ ID NO: 34) | 20 mg | 1 |
| Pre-imm. antiserum, Rabbit | E14838, E14839 | 100 μL | 2 |
| Small scale antibody, | E14838, E14839 | 100 μL | 2 |
| Rabbit | | | |
| Large scale antibody, | E14838 | 900 μL | 1 |
| Rabbit | | | |
| Large scale antibody, | E14839 | 800 μL | 1 |
| Rabbit | | | |
| Naked peptide | CLNSSEGVPDLLV (SEQ ID NO: 35) | 20 mg | 1 |
| Pre-imm. antiserum, Rabbit | E14840, E14841 | 100 μL | 2 |
| Small scale antibody, | E14840, E14841 | 100 μL | 2 |
| Rabbit | | | |
| Large scale antibody, | E14840, E14841 | 800 μL | 2 |
| Rabbit | | | |
| Naked peptide | CDSKPPLPPDEWV (SEQ ID NO: 36) | 20 mg | 1 |
| Pre-imm. antiserum, Rabbit | E14842, E14843 | 100 μL | 2 |
| Small scale antibody, | E14842, E14843 | 100 μL | 2 |
| Rabbit | | | |
| Large scale antibody, | E14842 | 1000 μL | 1 |
| Rabbit | | | |
| Large scale antibody, | E14843 | 800 μL | 1 |
| Rabbit | | | |
| Naked peptide | CDVKPPVLDVDDV (SEQ ID NO: 37) | 20 mg | 1 |
| Pre-imm. antiserum, Rabbit | E14844, E14845 | 100 μL | 2 |
| Small scale antibody, | E14844, E14845 | 100 μL | 2 |
| Rabbit | | | |
| Large scale antibody, | E14845 | 900 μL | 1 |
| Rabbit | | | |
| Large scale antibody, | E14844 | 800 μL | 1 |
| Rabbit | | | |
| Naked peptide | CEVKPPVLDVDDV (SEQ ID NO: 38) | 20 mg | 1 |
| Pre-imm. antiserum, Rabbit | E14846, E14847 | 100 μL | 2 |
| Small scale antibody, | E14846, E14847 | 100 μL | 2 |
| Rabbit | | | |
| Large scale antibody, | E14847 | 1000 μL | 1 |
| Rabbit | | | |
| Large scale antibody, | E14846 | 800 μL | 1 |
| Rabbit | | | |
| Naked peptide | CDVKPPVLDVDDV (SEQ ID NO: 37) | 20 mg | 1 |
| Pre-imm. antiserum, Rabbit | E14848, E14849 | 100 μL | 2 |
| Small scale antibody, | E14848, E14849 | 100 μL | 2 |
| Rabbit | | | |
| Large scale antibody, | E14849 | 1300 μL | 1 |
| Rabbit | | | |
| Large scale antibody, | E14848 | 900 μL | 1 |
| Rabbit | | | |
| Naked peptide | CEMRLPLLEVDDI (SEQ ID NO: 39) | 20 mg | 1 |
| Pre-imm. antiserum, Rabbit | E14850, E14851 | 100 μL | 2 |
| Small scale antibody, | E14850, E14851 | 100 μL | 2 |
| Rabbit | | | |
| Large scale antibody, | E14851 | 1200 μL | 1 |
| Rabbit | | | |
| Large scale antibody, | E14850 | 800 μL | 1 |
| Rabbit | | | |
| Naked peptide | CEHVIPSTLDDLI (SEQ ID NO: 40) | 20 mg | 1 |
| Pre-imm. antiserum, Rabbit | E14852, E14853 | 100 μL | 2 |
| Small scale antibody, | E14852, E14853 | 100 μL | 2 |
| Rabbit | | | |
| Large scale antibody, | E14853 | 1000 μL | 1 |
| Rabbit | | | |
| Large scale antibody, | E14852 | 800 μL | 1 |
| Rabbit | | | |
| Naked peptide | CNFQDVQRDKLYS (SEQ ID NO: 41) | 20 mg | 1 |
| Pre-imm. antiserum, Rabbit | E14854, E14855 | 100 μL | 2 |
| Small scale antibody, | E14854, E14855 | 100 μL | 2 |
| Rabbit | | | |
| Large scale antibody, | E14855 | 1000 μL | 1 |
| Rabbit | | | |
| Large scale antibody, | E14854 | 1200 μL | 1 |
| Rabbit | | | |
| Naked peptide | NEFPKNGWKNGC (SEQ ID NO: 21) | 20 mg | 1 |
| Pre-imm. antiserum, Rabbit | E14856, E14857 | 100 μL | 2 |
| Small scale antibody, | E14856, E14857 | 100 μL | 2 |
| Rabbit | | | |
| Large scale antibody, | E14857 | 900 μL | 1 |
| Rabbit | | | |
| Large scale antibody, | E14856 | 800 μL | 1 |
| Rabbit | | | |

### Example 4: Early Detection of Coronavirus

Subjects: Human subjects, females at age 62 and age 55, males at age 45 and 40. All the human subjects had no signs or symptoms of coronavirus infection.

Biomark ELISA Assay for Antibody:

Methods: For early detection of coronaviral infection, saliva samples from the human subjects were taken and then tested for the presence of antibodies in the subjects against a coronavirus, such as COVID-19, by a Biomark ELISA Assay using a peptide described below. The saliva samples of the human subjects who had no signs or symptoms of coronavirus infection were used as control subjects or control samples to determine the quantity of the antibody against 4 antigen peptides of SEQ ID NOs: 22-25.

Saliva samples were prepared by centrifuging at 3000 g for 5 minutes to collect the supernatant. The supernatant from saliva sample was diluted to 1:10 in 1% TB ST (100 μl of supernatant was diluted in 900 ul of TBST). The diluted samples were kept at room temperature. The procedure of the ELISA assay is as follows:
1. Fc fragment IgG (Goat anti-rabbit IgG Fc fragment secondary antibody) plates were made ahead of time by plating 100 μl of 500 ng IgG (25 ul of Fc IgG was added to 10 ml of Bicarbonate buffer) and stored at 4° C. (minimum Time for coating six hours).
2. The plate was washed 1× with 1% TBST prior to use for the experiment to remove any excess IgG.
3. Plate was then blocked with 1% BSA in PBS for 30 minutes at room temperature followed by washing 1× with TBST.
4. 100 μl of diluted saliva sample (or serum containing antibody) were added to the plates, then was add 50 μl of peptide SEQ ID NO.22 (or other peptide) with enzyme conjugates (HRP) (SEQ ID NO.22 HRP peptide was prepared by adding 156.25 μl of 100 ng HRP peptide to 2500 μl of 1% TBST). The plate was immobilized on shaker for 1 hour.
5. The plate was washed 3× with 1% TBST.
6. 100 μl of TMB were added to the plate and let it react for 20 minutes.
7. The reaction was stopped with 50 μl of 1N H₂SO₄
8. The plate was read at 450 nm.

Results: As shown in Table 5, all the numbers represent the absorbance readout at 450 nm from the ELISA and are duplicated. These numbers can be converted to the quantity (ng) of the antibody using a standard curve.

TABLE 5
ELISA Results

| Human | SEQ ID NO: | SEQ ID NO: | SEQ ID NO: | SEQ ID NO: |
|---|---|---|---|---|
| Subject | 22 | 23 | 24 | 25 |
| 62F | 1.1614/1.168 | 1.1867/1.2754 | 0.0399/0.0391 | 0.1901/0.191 |
| 55F | 1.1272/1.0446 | 1.9742/2.034 | 0.0479/0.0456 | 0.3111/0.4765 |
| 45M | 0.8186/0.8038 | 1.6574/1.3015 | 0.0567/0.0514 | 0.1835/0.2142 |
| 40M | 0.838/0.8878 | 1.0992/0.9947 | 0.0452/0.0419 | 0.1858/0.2112 |

Biomark ELISA Assay for Antigen:

The saliva samples from the human subjects can also be tested by a Biomark ELISA Assay for the presence of an antigen associated with a coronavirus, such as COVID-19, using an antibody according to an embodiment of the application. Saliva samples were prepared by centrifuging at 3000 g for 5 minutes to collect the supernatant. The supernatant from saliva sample was diluted to 1:10 in 1% TBST (100 μl of supernatant was diluted in 900 ul of TBST). The diluted samples were kept at room temperature. The procedure of the ELISA assay is as follows:
1. Fc fragment IgG (Goat anti-rabbit IgG Fc fragment secondary antibody) plates were made ahead of time by plating 100 μl of 500 ng IgG (25 ul of Fc IgG was added to 10 ml of Bicarbonate buffer) and stored at 4° C. (minimum Time for coating six hours).
2. The plate was washed 1× with 1% TBST prior to use for the experiment to remove any excess IgG.
3. Plate was then blocked with 1% BSA in PBS for 30 minutes at room temperature followed by washing 1× with TBST.
4. 100 μl of diluted saliva sample (or serum containing antibody) were added to the plates, then was add 50 μl of antibody developed against SEQ ID NO.22 (or other peptide) with enzyme conjugates (HRP). The plate was immobilized on shaker for 1 hour.
5. The plate was washed 3× with 1% TBST.
6. 100 μl of TMB were added to the plate and let it react for 20 minutes.
7. The reaction was stopped with 50 μl of 1N H₂SO₄
8. The plate was read at 450 nm.

### Example 5: Early Detection of Coronavirus

Subjects: Human subjects, females at age between 50-88, males at age between 31-64. All the human subjects had no signs or symptoms of coronavirus infection.

Methods: Saliva samples from the human subjects were taken and then tested by the Biomark ELISA Assay for Antibody described above to determine the quantity of the antibody against the peptide of SEQ ID NO: 27.

Results: As shown in Table 6, all the numbers represent the absorbance readout at 450 nm from the ELISA and are duplicated. These numbers can also be converted to the quantity (ng) of the antibody using a standard curve.

TABLE 6
ELISA Results Against Peptide of SEQ ID NO: 27

| Human | |
|---|---|
| Subject | Readout |
| Chris 31M | 0.5395/0.5518 |
| KY 31M | 0.1017/0.0922 |
| PT 51M | 0.1355/0.1089 |
| IS 31M | 0.143/0.1145 |
| TR 64M | 1.5433/1.5428 |
| TK 50F | 0.2393/0.2355 |
| RS 55F | 0.1161/0.0909 |
| HY 81F | 1.1273/1.0089 |
| RY 88M | 0.391/0.4123 |
| ED 87M | 1.8516/1.9387 |

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

We claim:

1. An antibody produced from a peptide of coronavirus envelop protein in animals for the detection or treatment of viral infection.

2. The antibody of claim 1, wherein the peptide is selected from the group consisting of:
(SEQ ID NO: 6)
MYSFVSEETGTLIVNSRVKNLNSSEGVPDLLV,
(SEQ ID NO: 2)
MYSFVSEETGTLIVNS,
(SEQ ID NO: 3)
RVKNLNSSEGVPDLLV,
(SEQ ID NO: 4)
NIVNVSLVKPTVYVYS,
(SEQ ID NO: 5)
FLLVTLAILTALRLC,
(SEQ ID NO: 6)
MYSFVSEETGTLIVNSRVKNLNSSEGVPDLLV,
(SEQ ID NO: 7)
IKAYNPDEALLV,
(SEQ ID NO: 8)
IKAYNPDGALLV,
(SEQ ID NO: 9)
IKAYNPDGDLLV,
(SEQ ID NO: 10)
IKAYNPDEAFLV,
(SEQ ID NO: 11)
HIDPFPKRVIDF,
(SEQ ID NO: 12)
RIDPLPSTVIDV,
(SEQ ID NO: 13)
QIAPVPAEVLNV,
(SEQ ID NO: 14)
LNSSEGVPDLLV,
(SEQ ID NO: 15)
DSKPPLPPDEWV,
(SEQ ID NO: 16)
DVKPPVLDVDDV,
(SEQ ID NO: 17)
DEKPPVLDVDDV,
(SEQ ID NO: 18)
EMRLPLLEVDDI,
(SEQ ID NO: 19)
EHVIPSTLDDLI,
(SEQ ID NO: 20)
NFQDVQRDKLYS,
and
(SEQ ID NO: 21)
NEFPKNGWKNGC.

3. The antibody of claim 1, wherein the peptide is selected from the group consisting of
(SEQ ID NO: 22)
CMYSFVSEETGTLIVNS,
(SEQ ID NO: 23)
CRVKNLNSSEGVPDLLV,
(SEQ ID NO: 24)
CNIVNVSLVKPTVYVYS,
(SEQ ID NO: 25)
KKFLLVTLAILTALRLC,
(SEQ ID NO: 26)
CMYSFVSEETGTLIVNSRVKNLNSSEGVPDLLV,
(SEQ ID NO: 27)
CYSFVSEETGTLIVNSRVKNLNSSEGVPDLLV,
(SEQ ID NO: 28)
CIKAYNPDEALLV,
(SEQ ID NO: 29)
CIKAYNPDGALLV,
(SEQ ID NO: 30)
CIKAYNPDGDLLV,
(SEQ ID NO: 31)
CIKAYNPDEAFLV,
(SEQ ID NO: 32)
CHIDPFPKRVIDF,
(SEQ ID NO: 33)
CRIDPLPSTVIDV,
(SEQ ID NO: 34)
CQIAPVPAEVLNV,
(SEQ ID NO: 35)
CLNSSEGVPDLLV,
(SEQ ID NO: 36)
CDSKPPLPPDEWV,
(SEQ ID NO: 37)
CDVKPPVLDVDDV,
(SEQ ID NO: 38)
CEVKPPVLDVDDV,
(SEQ ID NO: 39)
CEMRLPLLEVDDI,
(SEQ ID NO: 40)
CEHVIPSTLDDLI,
(SEQ ID NO: 41)
CNFQDVQRDKLYS,
and
(SEQ ID NO: 42)
CNEFPKNGWKNGC.

4. The antibody of claim 1, wherein the peptide is selected from the group consisting of SEQ IDs NO:2˜6 and SEQ IDs NO:22˜27.

5. The antibody of claim 1, wherein the peptide is selected from the group consisting of SEQ ID NO:2, SEQ ID NO:3, SEQ ID NO:6, SEQ ID NO:22, SEQ ID NO:23, SEQ ID NO:26, and SEQ ID NO:27.

6. The antibody of claim 1, wherein the peptide is
(SEQ ID NO: 43)
R₁-MYSFVSEETGTLIVNSRVKNLNSSEGVPDLLV-R₂,
wherein R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; and R₄ is H, OH, an alkyl, aralkyl, aryl or acyl radical having up to 19 carbon atoms; preferably R₂ is selected from the group consisting of OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc, and (H₃C)₂N(C═N)N(CH₃)₂.

7. The antibody of claim 1, wherein the peptide is
(SEQ ID NO: 44)
N-Ac-MYSFVSEETGTLIVNSRVKNLNSSEGVPDLLV-NH₂.

8. The antibody of claim 1, wherein the animal is selected from the group consisting of rabbit, dog, monkey, chimpanzee and human.

9. The antibody of claim 1, wherein the animal is selected from the group consisting of rabbit and human.

10. The antibody of claim 1, wherein the animal is selected from the rabbit.

11. The antibody of claim 1, wherein the detection is from saliva or nasal swab by using Biomark ELISA Assay.

12. A vaccine produced from a peptide of coronavirus envelop protein in animals for the treatment of viral infection.

13.-21. (canceled)

22. A method of detecting or treating a viral infection in a subject in need thereof, the method comprising administering an effective amount of an antibody produced from a peptide of coronavirus envelope protein in animals.

23. The method of claim 22, wherein the peptide is selected from the group consisting of:
(SEQ ID NO: 6)
MYSFVSEETGTLIVNSRVKNLNSSEGVPDLLV,
(SEQ ID NO: 2)
MYSFVSEETGTLIVNS,
(SEQ ID NO: 3)
RVKNLNSSEGVPDLLV,
(SEQ ID NO: 4)
NIVNVSLVKPTVYVYS,
(SEQ ID NO: 5)
FLLVTLAILTALRLC,
(SEQ ID NO: 6)
MYSFVSEETGTLIVNSRVKNLNSSEGVPDLLV,
(SEQ ID NO: 7)
IKAYNPDEALLV,
(SEQ ID NO: 8)
IKAYNPDGALLV,
(SEQ ID NO: 9)
IKAYNPDGDLLV,
(SEQ ID NO: 10)
IKAYNPDEAFLV,
(SEQ ID NO: 11)
HIDPFPKRVIDF,
(SEQ ID NO: 12)
RIDPLPSTVIDV,
(SEQ ID NO: 13)
QIAPVPAEVLNV,
(SEQ ID NO: 14)
LNSSEGVPDLLV,
(SEQ ID NO: 15)
DSKPPLPPDEWV,
(SEQ ID NO: 16)
DVKPPVLDVDDV,
(SEQ ID NO: 17)
DEKPPVLDVDDV,
(SEQ ID NO: 18)
EMRLPLLEVDDI,
(SEQ ID NO: 19)
EHVIPSTLDDLI,
(SEQ ID NO: 20)
NFQDVQRDKLYS,
and
(SEQ ID NO: 21)
NEFPKNGWKNGC.

24. The method of claim 22, wherein the peptide is selected from the group consisting of
(SEQ ID NO: 22)
CMYSFVSEETGTLIVNS,
(SEQ ID NO: 23)
CRVKNLNSSEGVPDLLV,
(SEQ ID NO: 24)
CNIVNVSLVKPTVYVYS,
(SEQ ID NO: 25)
KKFLLVTLAILTALRLC,
(SEQ ID NO: 26)
CMYSFVSEETGTLIVNSRVKNLNSSEGVPDLLV,
(SEQ ID NO: 27)
CYSFVSEETGTLIVNSRVKNLNSSEGVPDLLV,
(SEQ ID NO: 28)
CIKAYNPDEALLV,
(SEQ ID NO: 29)
CIKAYNPDGALLV,
(SEQ ID NO: 30)
CIKAYNPDGDLLV,
(SEQ ID NO: 31)
CIKAYNPDEAFLV,
(SEQ ID NO: 32)
CHIDPFPKRVIDF,
(SEQ ID NO: 33)
CRIDPLPSTVIDV,
(SEQ ID NO: 34)
CQIAPVPAEVLNV,
(SEQ ID NO: 35)
CLNSSEGVPDLLV,
(SEQ ID NO: 36)
CDSKPPLPPDEWV,
(SEQ ID NO: 37)
CDVKPPVLDVDDV,
(SEQ ID NO: 38)
CEVKPPVLDVDDV,
(SEQ ID NO: 39)
CEMRLPLLEVDDI,
(SEQ ID NO: 40)
CEHVIPSTLDDLI,
(SEQ ID NO: 41)
CNFQDVQRDKLYS,
and
(SEQ ID NO: 42)
CNEFPKNGWKNGC.

25. The method of claim 22, wherein the peptide is selected from the group consisting of SEQ IDs NO:2˜6 and SEQ IDs NO:22˜27.

26. The method of claim 22, wherein the peptide is selected from the group consisting of SEQ ID NO:2, SEQ ID NO:3, SEQ ID NO:6, SEQ ID NO:22, SEQ ID NO:23, SEQ ID NO:26, and SEQ ID NO:27.

27. The method of claim 22, wherein the peptide is
(SEQ ID NO: 43)
R₁-MYSFVSEETGTLIVNSRVKNLNSSEGVPDLLV-R₂,
wherein R₁ is an acyl radical having up to 29 carbon atoms; R₂ is OR₃, NHR₄, or any amino group containing radical having up to 10 carbon atoms; R₃ is H, an alkyl, aralkyl or aryl radical having up to 19 carbon atoms; and R₄ is H, OH, an alkyl, aralkyl, aryl or acyl radical having up to 19 carbon atoms; preferably R₂ is selected from the group consisting of OH, OEt, NHOH, NH₂, NHNH₂, NHNHAc, NHCONH₂, NH(C═NH)NH₂, NH(C═NH)NHNH₂,NHNH(C═NH)NH₂, NH(C═NH)NHNHAc, NHNH(C═NH)NHAc, and (H₃C)₂N(C═N)N(CH₃)₂.

28. The method of claim 22, wherein the peptide is
(SEQ ID NO: 44)
N-Ac-MYSFVSEETGTLIVNSRVKNLNSSEGVPDLLV-NH₂.

29. The method of claim 22, wherein the animal is selected from the group consisting of rabbit, dog, monkey, chimpanzee and human.

30.-42. (canceled)
